(19) United States
(12) Patent Application Publication
(10) Pub. No.: US 2023/0348997 A1
(43) Pub. Date: Nov. 2, 2023

(54) SIGNATURES IN CELL-FREE DNA TO DETECT DISEASE, TRACK TREATMENT RESPONSE, AND INFORM TREATMENT DECISIONS

(71) Applicant: THE REGENTS OF THE UNIVERSITY OF COLORADO, A BODY CORPORATE, AURORA, CO (US)

(72) Inventors: PETER KABOS, GREENWOOD VILLAGE, CO (US); SRINIVAS RAMACHANDRAN, DENVER, CO (US); ALEXIS ZUKOWSKI, DENVER, CO (US); SATYANARAYAN RAO, AURORA, CO (US); AMY HAN, AURORA, CO (US)

(21) Appl. No.: 18/245,749

(22) PCT Filed: Sep. 17, 2021
(86) PCT No.: PCT/US2021/050819

§ 371 Date: Mar. 17, 2023

Related U.S. Application Data
(60) Provisional application No. 63/079,589, filed on Sep. 17, 2020.
(60) Provisional application No. 63/124,179, filed on Dec. 11, 2020.

(51) Int. Cl. C12Q 1/6886

(52) U.S. Cl. CPC C12Q 1/6886; C12Q 2600/158

(57) ABSTRACT

Provided by the inventive concept are methods and materials for analyzing cell-free DNA (cfDNA), such as analyzing cfDNA to determine transcription factor (TF) binding, and/or gene expression in order to detect disease, track treatment response of disease, and inform treatment decisions of disease, such as to detect, track treatment response of, and inform treatment decisions for cancer.

### CROSS-REFERENCE TO RELATED APPLICATIONS

The present application claims the benefit of U.S. Provisional Patent Application No. 63/079,589, filed Sep. 17, 2020, and U.S. Provisional Patent Application No. 63/124,179, filed Dec. 11, 2020, the disclosures of each of which are incorporated herein by reference in their entireties.

### FIELD

The present inventive concept is related to methods of detecting and treating disease, such as cancers and inflammatory diseases, and tracking treatment response and/or recurrence of disease through analysis of cell-free DNA (cfDNA).

### BACKGROUND

The current capability for real time monitoring of patients with solid tumors is limited to analysis of blood counts, electrolytes, liver and kidney function. For example, in breast cancer patients, the closest approach to real time monitoring of the disease is by measuring carcinoembryonic (CEA) and mucin 1 antigens (CA27-29, CA15-3) levels in serum. These data give limited information on treatment response. Given a more widespread use of targeted agents, mutational analysis is being adopted more readily. However, many cancers do not have a high mutation load, and once a mutation is detected, limited if any further information can be gained by repeating the analysis. In addition, the detection of specific mutations is often limited to biopsies typically performed only once during the entire course of treatment of metastatic disease.

There is mounting evidence that specialized cell-free DNA (cfDNA) analysis can add information that is personalized, specific to disease state, and has potential to deliver essential insight for clinical decision making. In healthy individuals, most cfDNA is generated by normal turnover of lymphoid and myeloid tissue. In individuals with cancer, tumor cells contribute significantly to the cfDNA content. In addition to providing fragments of DNA sequences from their cells of origin, cfDNA provides information about the chromatin structure in these cells. This is because cfDNA is the result of the action of endogenous nucleases on DNA that is not protected by proteins such as nucleosomes or transcription factors (TFs) that were bound to the genome in cells of origin.

### SUMMARY

Thus, when analyzed appropriately, cfDNA sequencing data can be used to noninvasively track cancer state by assessing TF binding patterns. Aspects of the inventive concept relate to leveraging TF binding patterns contained in cfDNA, which is currently untapped, to provide a novel experimental and data analysis pipeline than may be used to report on real time disease status, such as in malignant disease, for example, breast cancer and prostate cancer, and inflammatory states. Further aspects of the inventive concept include a custom developed panel of TF binding sites (TFBS) that can cost effectively and non-invasively track both disease state, treatment efficacy, and offer personalized information when change in treatment is indicated. The same approach can be applied by tracking immune specific TFs, in inflammatory diseases.

According to an aspect of the inventive concept, provided is a method of identifying a disease state in a subject including: sequencing of cell-free DNA (cfDNA) derived from the subject; obtaining a map of transcription factor (TF) binding sites; obtaining a map of subnucleosomes at promoters associated with the map of TF binding sites; and determining whether the subject has the disease or disorder if the map of subnucleosomes at promoters associated with the map of TF binding sites for the subject matches a signature for an individual having the disease or disorder. Also provided is a method of treating a disease or disorder including: sequencing of cell-free DNA (cfDNA) derived from the subject; obtaining a map of transcription factor (TF) binding sites; obtaining a map of subnucleosomes at promoters associated with the map of TF binding sites; and determining whether the subject has the disease or disorder if the map of subnucleosomes at promoters associated with the map of TF binding sites for the subject matches a signature for an individual having the disease or disorder, and treating the subject if it is determined that the subject has the disease or disorder.

According to another aspect of the inventive concept, provided is a method of monitoring efficacy or progress of treatment for a disease in a subject in need thereof including: sequencing of cell-free DNA (cfDNA) derived from a subject undergoing treatment for a disease or disorder; obtaining a map of transcription factor (TF) binding sites; obtaining a map of subnucleosomes at promoters associated with the map of TF binding sites; and determining whether treatment of the subject is effective if the map of subnucleosomes at promoters associate with the map of TF binding sites for the subject matches a signature for an individual that is free of the disease or disorder.

According to yet another aspect of the inventive concept, provided is a method of monitoring recurrence of a disease or disorder in a subject in need thereof including: sequencing of cell-free DNA (cfDNA) derived from the subject; obtaining a map of TF binding sites and subnucleosomes at promoters associated with the TF binding sites from the sequencing of the cfDNA; and determining whether the subject is having a recurrence of the disease or disorder if the map of subnucleosomes at promoters associated and TF binding sites for the subject matches a signature for an individual having the disease or disorder. Also provided is a method of treating recurrence of a disease or disorder including: sequencing of cell-free DNA (cfDNA) derived from the subject; obtaining a map of TF binding sites and subnucleosomes at promoters associated with the TF binding sites from the sequencing of the cfDNA; and determining whether the subject is having a recurrence of the disease or disorder if the map of subnucleosomes at promoters associated and TF binding sites for the subject matches a signature for an individual having the disease or disorder, and treating the subject for the disease or disorder if it is determined that the subject is having a recurrence of the disease or disorder.

According to yet another aspect of the inventive concept, provided is a method of identifying cellular origin or origins of cfDNA from a subject including: sequencing of cell-free DNA (cfDNA) derived from the subject; obtaining a map of TF binding sites; obtaining a map of subnucleosomes at promoters associated with TF binding sites from the sequencing of the cfDNA; and determining the cellular origin or origins of the cfDNA from the map of subnucleosomes at promoters and TF binding sites, wherein a TF binding signature, or mixtures thereof, to which the map of subnucleosomes at promoters and TF binding sites matches is indicative of the cellular origin or origins of the cfDNA from the subject.

According to yet another aspect of the inventive concept, provided is a method for obtaining a signature for cellular origin of cfDNA comprising: sequencing cfDNA derived from a sample; and obtaining a map of subnucleosomes at promoters associated with a set of TF binding sites, to provide a signature for cellular origin of the cfDNA in the sample.

Also provided are kits to perform any of the methods and aspects of the inventive concept as set forth herein.

### BRIEF DESCRIPTION OF THE DRAWINGS

FIG. 1. Workflow for classifying TFBS according to the cfDNA length distribution. The expected fragment sizes for each cluster is indicated in parentheses.

FIGS. 2A-2D. Detection of CTCF binding in healthy plasma. FIG. 2A: Clustering of length distribution of fragments at CTCF binding sites. FIG. 2B: Enrichment of short footprints at CTCF binding sites genome-wide. The sites are arranged according to clusters in FIG. 2A, with cluster 1 at the top and cluster 6 at the bottom. Clusters 1 and 2 show strong TF footprint in cfDNA. FIG. 2C: Enrichment of nucleosomal footprints in the same order of CTCF sites as FIG. 2B. Strong phasing of nucleosomes upstream and downstream of CTCF sites for clusters 1 and 2 is observed. FIG. 2D: ChIP-seq scores at CTCF sites for different clusters from a lymphoid cell line. Clusters 1 and 2 have sites with significantly higher ChIP scores compared to other clusters.

FIGS. 3A-3D. Detection of PU.1 binding in healthy plasma. FIG. 3A: Enrichment of short footprints at PU.1 binding sites genome-wide. The sites are arranged according to expected length of fragment clusters, with cluster 1 at the top and cluster 6 at the bottom. Clusters 1 and 2 show strong TF footprint in cfDNA. FIG. 3B: Enrichment of nucleosomal footprints in the same order of PU.1 sites as FIG. 3A. Strong phasing of nucleosomes upstream and downstream of PU.1 sites for clusters 1 and 2 is observed. FIG. 3C: ChIP-seq scores at PU.1 sites for different clusters from a lymphoid cell line. Clusters 1, 2, and 3 have sites with significantly higher ChIP scores compared to other clusters. FIG. 3D: Enrichment of short fragments and nucleosomal fragments that aligned to the human genome in cfDNA datasets from two PDX models, plotted in the same order as FIG. 3A. Complete lack of enrichment of short fragments and phasing of nucleosomal fragments is observed at PU.1 binding sites, showing lack of PU.1 binding in tumor.

FIGS. 4A-4E. Tumor-specific FOXA1 footprints. FIG. 4A: Clustering of length distribution of fragments at FOXA1 binding sites from healthy plasma. Note that only one cluster has short fragments (cluster 1). FIG. 4B: Clustering of length distribution of fragments at FOXA1 binding sites from PDX plasma. Note that most clusters are enriched for short fragments (except cluster 6). ChIP-seq scores at FOXA sites for different clusters from MCF-7 cells for clusters in healthy plasma (FIG. 4C), and PDX plasma (FIG. 4D). Clusters from healthy plasma have no significant differences in ChIP scores, whereas clusters from PDX plasma with short footprints have significantly higher scores than the cluster with nucleosomal footprint (cluster 6, FIG. 4D). FIG. 4E: Enrichment of short footprints at FOXA1 binding sites genome-wide from PDX plasma. The sites are arranged according to clusters in FIG. 4B, with cluster 1 at the top and cluster 6 at the bottom. Clusters 1 and 2 show strong TF footprint in cfDNA.

FIGS. 5A-5C. Tumor-specific ER footprints. FIG. 5A: Enrichment of short footprints at ER binding sites genome-wide as determined by CUT&RUN. The sites are arranged according to expected length of fragment clusters, with cluster 1 at the top and cluster 6 at the bottom. Clusters 1 and 2 show strong TF footprint in cfDNA. FIG. 5B: Enrichment of nucleosomal footprints in the same order of ER sites as FIG. 5A. FIG. 5C: CUT&RUN scores at ER binding sites for different clusters from MCF7. Clusters 1-5 have sites with significantly higher ChIP scores compared to cluster 6 and the median score of the clusters correlates with the cluster number.

FIG. 6. TFBS length clusters are disease-state specific. Ratio of observed overlap between cfDNA length clusters between two PDX models to the expected overlap based on chance. The high ratios between Cluster 1 of MCF7 (Cl1) and Clusters 2, 3, and 4 of PT65 (Cl2, Cl3, Cl4) indicate that the top ER-binding sites in MCF7 overlap with lower ER binding sites in PT65. In other words, there is a shift in ER TFBS that are enriched for short protections in plasma from MCF7 to PT65, indicating that cfDNA TF profiles are disease-state specific. The number of peaks used in this analysis are 6827. The statistical significance is expressed using the following key: *: 0.05<=p-val<0.010, **: 0.001<=p-val<0.050, ***: 0.0001<=p-val<0.001, ****: p-val<0.0001.

FIG. 7. Design of tiled probes spanning promoter sequences of 13,000 genes.

FIG. 8. Enrichment of pooled SSP libraries over unenriched libraries prior to sequencing.

FIG. 9. Identification of promoter nucleosomes from promoter enriched libraries compared to unenriched libraries.

FIG. 10. Schematic for identifying subset of binding sites with TF footprints. e) When TFs or nucleosomes are bound at TF binding sites, they protect different lengths of DNA from nucleases in dying cells in the human body. Panel B) When sequenced cfDNA fragments are mapped to TFBSs±50 bp, varying numbers of short and long cfDNA fragments are found at the three TFBSs shown in (panel A). Panel C) cfDNA fragment length distribution is estimated at each TFBS (purple bars) and smoothed using kernel density estimation (green line). Panel D) K-means clustering is performed on smoothed length distribution to group TFBSs with similar cfDNA fragment length distribution. Here, smoothened length distributions of clusters of CTCF TFBS are shown. Weighted length (W.L.) for each CTCF length cluster is shown in parentheses.

FIG. 11. cfDNA maps CTCF-nucleosome dynamics in plasma from a healthy individual. Panel A) Enrichment over the mean signal in TFBS±1 Kb of cfDNA short (<80 bp) fragments is plotted as a heatmap (top, 117,144 CTCF TFBS) and as metaplots for each cluster (bottom). Panel B) Same as (panel A) for nucleosome-sized fragments (130-180 bp). Panel C) Same as (panel B) for MNase-seq dataset from GM12878 cells. Panel D) Fragment midpoint versus fragment length plot (V-plot) of cfDNA fragments centered at CTCF binding sites from clusters 1 and 2. Fragment densities at motif center±500 bp (top) and motif center±200 bp (bottom) are plotted. Panel E) Boxplot of CTCF mean ChIP signal from the GM12878 cell line across length clusters. Number of sites (n) in length clusters and p-value using Kolmogorov-Smirnov (KS) test with alternative=“greater” option are: Cl1: n=11978, p(1,6)<2.2×10⁻¹⁶; Cl2: n=12811, p(2,6)<2.2×10⁻¹⁶; Cl3: n=28132, p(3,6)=1.1×10⁻³¹; Cl4: n=20839, p(4,6)=0.95; Cl5: n=22087, p(5,6)=0.96; Cl6: n=21297. p(a,b) denotes p-values calculated between scores in length cluster “a” and scores in length cluster “b”. Significance string (****) is added if p<0.0001 after Bonferroni correction.

FIG. 12. cfDNA of lymphoid/myeloid origin contains hematopoietic TF footprints. Panel A) Enrichment over the mean signal in PU.1 TFBS±1 Kb of cfDNA short (<80 bp) fragments is plotted as a heatmap (top, 53,613 PU.1 TFBS) and as metaplots for each cluster (bottom). Panel B) Same as (panel A) for nucleosome-sized fragments (130-180 bp). Panel C) Boxplot of PU.1 mean ChIP signal (Log 2) from GM12878 cell line across length clusters. Number of sites (n) in length clusters and p-value using KS test are: Cl1:n=6528, p(1,6)=9.2×10⁻²⁰; Cl2: n=6447, p(2,6)=1.7×10⁻²²; Cl3: n=10377, p(3,6)=0.00011; Cl4: n=10036, p(4,6)=0.19; Cl5: n=9673, p(5,6)=0.7; Cl6: n=10552. Significant string was determined after Bonferroni correction. Panel D) Enrichment metaplots for short fragments in PU.1 TFBS belonging to clusters 1 and 2 for healthy (IH02), cancer (IC15, 17, 20, 35, and 37) cfDNA and PDX cfDNA (MCF7 and UCD65). Panel E) Boxplot of mean of short fragment enrichment (TFBS±50 bp) for the samples and TFBS plotted in (panel D). e) Same as (panel A) for LYL1 (7,999 TFBS). Panel G) Same as (panel B) for LYL1. Panel H) Same as (panel C) for LYL1. Number of sites (n) in length clusters and p-value using KS test are: Cl1: n=1083, p(1,6)=4.7×10⁻¹²; Cl2: n=1001, p(2,6)=3×10⁻⁷; Cl3: n=1748, p(3,6)=0.18; Cl4: n=1351, p(4,6)=0.15; Cl5: n=1415, p(5,6)=0.62; Cl6: n=1401. Significant string was determined after Bonferroni correction. Panel I) Same as (panel D) for LYL1. Panel J) Same as (panel E) for LYL1. ****: p<0.0001, ***: 0.0001<p<0.001

FIG. 13. ER+ PDX models enable identification of pure tumor cfDNA footprints for ER. Panel A) Schematic of human tumor implant in mouse and the process of identifying tumor cfDNA by mapping mouse plasma cfDNA to an in silico concatenated genome. Fragments mapping uniquely to human (violet lines) defines tumor cfDNA (ctDNA). Fragments mapping uniquely to mouse genome (blue lines) arise from the tumor microenvironment and from the mouse lymphoid/myeloid cells. Fragments mapping to both genomes were discarded (green lines). Panel C) Enrichment over the mean signal in TFBS±1 Kb of cfDNA short (<80 bp) fragments is plotted as a heatmap (top, 83,311 ER TFBS) and as metaplots for each cluster (bottom). Panel D) Boxplot of ER CUT&RUN scores for peak summits in k-means clusters. Number of sites (n) in length clusters and p-value using KS test are: Cl1: n=12785, p(1,6)=1.2×10⁻¹⁵¹; Cl2: n=13301, p(2,6)=7.9×10⁻¹¹⁶; Cl3: n=11943, p(3,6)=1.5×10⁻⁸⁰; Cl4: n=10363, p(4,6)=1.6×10⁻³⁷; Cl5: n=10848, p(5,6)=1.1×10⁻⁰⁸; Cl6: n=24029. Significant string was determined after Bonferroni correction. ****: p<0.0001, ***: 0.0001<p<0.001

FIG. 14. ER+ PDX models enable identification of pure tumor cfDNA footprints for FOXA1. Panel A) Average length distributions at clusters of FOXA1 CUT&RUN peaks (summit±50 bp) generated by k-means clustering (n=6) of the ctDNA fragment length distribution. Panel B) Enrichment over the mean signal in TFBS±1 Kb of cfDNA short (<80 bp) fragments is plotted as a heatmap (top, 39,500 FOXA1 TFBS) and as metaplots for each cluster (bottom). Panel C) Boxplot of FOXA1 CUT&RUN scores (see methods) for peak summits in K-means clusters. p values from Kolmogorov-Smirnov test. Number of sites (n) in length clusters and p-value using KS test are: Cl1: n=4220, p(1,6)=3.4×10⁻³⁶; Cl2: n=5669, p(2,6)=3.2×10⁻¹⁹; Cl3: n=5699, p(3,6)=4.5×10⁻¹⁵; Cl4: n=4831, p(4,6)=3.1×10⁻¹⁰; Cl5: n=9033, p(5,6)=3.9×10⁻¹⁰; Cl6: n=10017. Significant string was determined after Bonferroni correction. ****: p<0.0001.

FIG. 15. Tissue-specific TF binding sites enable detection of disease states. Panel A) Upset plots (75) of cfDNA-inferred bound sites in different plasma samples for LYL1, PU.1, CTCF, FOXA1 and ER (left to right). Plots were generated using ComplexUpset R package (DOI: 10.5281/zenodo.4661589). Panel B) Boxplots of TF binding scores measured as mean enrichment of short fragments at CUT&RUN peak summit±100 bp for ER and FOXA1 and motif center±50 bp for LYL1, PU.1 and CTCF. CSS—cancer specific sites; HSS; healthy specific sites Panel C) Line plot of median t-statistic calculated for change in the binding scores (score in healthy plasma used as baseline) at binding sites of an individual TF or a collection of TFs at different in silico dilutions of healthy cfDNA with PDX ctDNA. At each dilution, 100 bootstrapped samples were generated. Horizontal dashed line is drawn where the t-statistic equals 5. Panel D) Boxplot of TF binding scores in pure ctDNA (UCD65/MCF7) at ER and FOXA1 sites specific to UCD65 or MCF7. Panel E) Boxplot of TF binding scores in pure ctDNA (UCD65/UCD4) at ER and FOXA1 sites specific to UCD4 against UCD65. Panel F) Boxplot of TF binding scores in pure ctDNA (MCF7/UCD4) at ER and FOXA1 sites specific to UCD4 against MCF7. Panel G) Line plot of median t-statistic calculated for the change in TF binding scores at UCD65 or MCF7-specific ER, FOXA1, or for ER and FOXA1 sites combined. Panel H) Same as (panel G) for UCD4-specific ER and FOXA1 sites against UCD65. Panel I) same as (panel G) for UCD4-specific ER and FOXA1 sites against MCF7.

FIG. 16. Plasma footprints represent TF specific accessibility in primary tumors and can predict presence of breast cancer Panel A) Heatmap of ATAC scores from BRCA cohorts from TCGA stratified based on ER expression levels (ER low: TPM<10, ER high: TPM≥10) at cfDNA-inferred ER CUT&RUN peaks with ER motif. The single column heatmap (left) plots the difference in mean ATAC scores between tumors with high ER expression and tumors with and low ER expression. The sites are ordered in ascending order of difference in ATAC scores between the two groups and the horizontal line separates sites with higher score in ER high compared to ER low. Panel B) Same as (panel A) for FOXA1 sites. Panel C) Heatmap of t-statistic calculated between tumors grouped by TF expression (columns; low (bottom 15 cohorts) and high (top 15 cohorts) expression levels) at binding sites of different TFs (rows). Panel D) Boxplot of mean ATAC-scores at ER sites (n=1,190) where tumors are stratified by both ER and FOXA1 expression. Panel E) Boxplot of mean ATAC-scores at FOXA1 sites (n=7,942) where patients are stratified by both ER and FOXA1 expression. Panel F) Heatmap of enrichment (Log 2 (Observed/Expected)) of frequency of TF features selected for a given classification (rows) divided by overall frequency of TF features. Panel G) Prediction accuracy of classifying patients to BC (breast cancer) and nonBC (non-breast cancer) using TF scores from plasma cfDNA using leave one out cross-validation.

FIG. 17. Subnucleosome enrichment predicts treatment response in non-small cell lung cancer (NSCLC). Panel A) Enrichment of 155-170 bp fragments from cfDNA extracted from NSCLC patient plasma mapped relative to TSS, averaged over gene expression quartilies of Neutrophils. Panel B) Boxplot of rank of adenocarcinoma average expression when compared to NSCLC cfDNA SE. Panel C) Similarity of NSCLC cfDNA SE (of responders and non-responders to anti-PD-1 therapy) to CD8⁺ T cell expression profile is calculated using Spearman correlation. Panel D) Enrichment of 155-170 bp fragments (nucleosomes) from cfDNA mapped relative to TSS of PD-1 gene. The nucleosome profiles were averaged over responders (n=10) and non-responders (n=11). The left arrow indicates promoter region. The arrow on the right shows position of the +1 nucleosome. Panel E) Fragments mapping to +1 nucleosome positions of PD-1 and PD-L1 were combined to calculate SE scores.

FIG. 18. CD8 T Cell TF footprints predict treatment response. cfDNA length clustering (k=6) at motifs inside published ATAC peaks identifies clusters with TF footprints in responders (top left) and non-responders (top right). The nucleosome distribution at these clusters shows depletion at motif and ordered nucleosome arrays upstream and downstream of the motifs, further confirming TF binding (bottom left and right).

FIG. 19. Immune TF footprints predict treatment response. Panel A) Heatmap of <60 bp cfDNA fragments shown for the subset of TF footprints that are predictive of treatment response (responders—top left and non-responders—top right). The corresponding metaplots of cfDNA nucleosome density relative to motif is shown below. Nucleosomes are depleted at motif and are phased relative to the binding site. Panel B) Scores at response-predictive sites for responders (n=10) and non-responders (n=11) shows striking separation.

### DETAILED DESCRIPTION

In the following detailed description, embodiments of the present inventive concept are described in detail to enable practice of the inventive concept. Although the inventive concept is described with reference to these specific embodiments, it should be appreciated that the inventive concept can be embodied in different forms and should not be construed as limited to the embodiments set forth herein. Rather, these embodiments are provided so that this disclosure will be thorough and complete, and will fully convey the scope of the inventive concept to those skilled in the art. All publications cited herein are incorporated by reference in their entireties for their teachings.

The inventive concept includes numerous alternatives, modifications, and equivalents as will become apparent from consideration of the following detailed description.

Unless otherwise defined, all technical terms used herein have the same meaning as commonly understood by one of ordinary skill in the art to which this disclosure belongs.

Current clinical approaches to identify disease states using cfDNA have been primarily limited to documenting disease-specific mutations. However, disease mutations provide limited information in regard to treatment resistance and response. According to embodiments of the inventive concept, by looking at ˜50,000 sites that reflect the functional state of, for example, a tumor, higher sensitivity, and diagnostic information compared to current approaches can be achieved. Furthermore, by enriching for the selected sites using hybridization techniques, sequencing costs can be substantially lowered.

Thus, analysis of circulating cell-free DNA (cfDNA) can provide a non-invasive means to detect a tumor at earlier stages than traditional diagnostic techniques. Most cfDNA in a healthy person is generated by normal turnover of lymphoid and myeloid tissue. From the onset of cancer, turnover of tumor cells also contributes to cfDNA. Thus, identifying the cells-of-origin of cfDNA can enable detection of disease. Current approaches identify tumor cells-of-origin of cfDNA by searching for cancer-specific mutations. These methods suffer from two major limitations: first, in early stages of disease, circulating mutant DNA is expected to be a minute fraction of cfDNA since most cfDNA comes from normal turnover of lymphoid and myeloid tissue. Second, the reference set of mutations to be screened is limited by current knowledge and the breadth of disease states. These mutations also occur naturally in healthy cells at low levels and in blood cells due to clonal hematopoesis. These limitations prevent cfDNA sequencing from being a reliable method for early diagnosis of cancer. Here, we propose to develop a method to identify cells-of-origin of cfDNA at higher sensitivity and lower cost compared to current approaches that will provide a robust and unbiased approach for detection of tumors.

Applications of the innovative aspects of the present inventive concept may include:
1. Early detection of cancer using a combination of signal enrichment, gene expression profile and disease specific TF binding sites;
2. Real time disease monitoring on therapy to help determine the extent of disease and distinguish response versus disease progression based on cfDNA profile;
5 3. Individualized care and patient selection based on accurate definition of specific disease states, and to switch therapy when appropriate (including immunotherapy);
4. Define and monitor systemic inflammatory states (i.e. inflammatory bowel disease, systemic lupus), based on immune footprint of cfDNA from lymphocytes, monocytes/macrophages and NK cells; TFs specific to disease states (i.e. EGR2 for M1 versus M2 state of macrophage differentiation) are used in combination with cell specific gene expression profile inferred from cfDNA analysis;
5. Treatment of disease, for example, based on detection of cancer as set forth in 1 and administering of treatment and/or therapy if indicated;
6. Assessing effectiveness of treatment of disease, for example, based on disease monitoring of treatment and/or therapy as set forth in 2, including adjusting of treatment and/or therapy, if indicated; and
7. Individualization of treatment and/or therapy, for example, based individualized care and patient selection as set forth in 3, including adjusting treatment and/or therapy, if indicated.

Accordingly, described herein are methods and materials for detecting a disease or disorder, tracking treatment response, and inform treatment decisions related to disease or a disorder. Embodiments of the inventive concept include analysis of cell-free DNA (cfDNA) derived from a subject. cfDNA was discovered as periodic fragments of genomic DNA generated by endogenous nucleases. However, it was described only recently that cfDNA represents an accurate map of the chromatin landscape of cells undergoing turnover. From this knowledge, a genome-wide map of nucleosome and TF binding of cells that gave rise to cfDNA can be reconstructed. In order to do so requires the ability to recover DNA fragments less than about 200 bp, for example, recovering DNA fragments of all lengths from about 40 to about 200 bp.

In embodiments of the inventive concept, analysis of cfDNA may include isolation of cfDNA and preparation of cfDNA libraries, such as sequencing libraries of cfDNA suitable for deep sequencing of cfDNA. Although the method of preparation of cfDNA libraries is not particularly limited and may be any method that would be appreciated by one of skill in the art, the method of library construction should effectively recover DNA fragments of less than about 200 bp, less than about 175 bp, less than about 160 bp, less than about 150 bp, less than about 140 bp, less than about 130 bp, less than about 120 bp, less than about 110 bp, less than about 100 bp, less than about 90 bp, less than about 80 bp, less than about 70 bp, less than about 60 bp, less than about 50 bp, and should recover DNA fragments down to about 40 bp in size, such as methods for preparing sequencing libraries from cfDNA that have been denatured into single stranded DNA, for example, a method as described by Snyder et al., 2016, Cell 164, 57-68 and/or a method described by Gansauge and Meyer, 2013, Nat. Protoc. 8, 737-748 the disclosures of which are incorporated herein by reference.

Although not particularly limited thereto, the source of the cfDNA for analysis according to the present inventive concept generally may be from blood and/or blood plasma derived from the subject. In some embodiments, cfDNA derived from the source may include selectively enriching for promoters, promoter sequences, and/or sequences associated with promoter sequences using oligonucleotides directed toward promoter sequences in cfDNA, for example, from the transcription start site (TSS)+about 300 bp downstream from the TSS, that retains accurate representation of the promoters in cfDNA while reducing sequencing cost.

In other embodiments of the inventive concept, cfDNA analysis may include deep sequencing of the cfDNA sequencing libraries. As with cfDNA sequencing library preparation, the method of deep sequencing is not particularly limited, and the method may be any that would be appreciated by one of skill in the art. In some embodiments, the method of deep sequencing is a next generation sequencing (NGS) method, for example, an NGS platform, such as available from Illumina, Ion Torrent, PacBio, Nanopore, and 10X Genomics. In some embodiments, sequencing may include pair-end Illumina sequencing, but is not limited thereto. In some embodiments, sequencing according to methods of the inventive concept can determine both location of a cfDNA fragment in the genome and length of the cfDNA fragment. According to embodiments of the inventive concept, sequencing of cfDNA, and through subnucleosome analysis at promoter regions, may provide a whole transcriptional profile of cells that give rise to the cfDNA, i.e., through a map of subnucleosomes associated with promoters, transcription factor (TF) binding sites and/or gene expression from the cells that give rise to the cfDNA from subnucleosome analysis at promoters. Methods of analyzing cfDNA for phenotypes are discussed in Zukowski et al. (2020) Open Biol. 10: 200119. dx.doi.org/10.1098/rsob.200119, the disclosures of which are incorporated herein by reference.

According to embodiments of the inventive concept, gene expression, or active transcription of genes, may include mapping of TF binding sites, and mapping of subnucleosomes at/associated with promoters among the mapped TF binding sites, more particularly, mapping of subnucleosomes at/associated with promoters among a set of TF binding sites, to obtain a map of transcriptionally active genes among the mapped TF binding sites. The method of mapping/selecting a set of TF binding sites, i.e., the TF binding sites at which subnucleosomes associated with promoters and/or the TF binding sites are mapped, is not particularly limited, and may be any that may be appreciated by one of skill in the art. For example, methods may include methods for characterizing protein-DNA interactions, such as MNase-seq, CATCH-IT, ChIP-seq, CUT&RUN, etc., or any combination thereof.

In some embodiments, mapping of TF binding sites, and mapping of subnucleosomes associated with/at promoters, such as at transcriptionally active promoters/TF binding sites may be performed, for example, by methods as described by Ramachandran et al., 2017, Mol. Cell 68, 1038-1052, and Supplemental Information for Ramachandran et al. contained at https://doi.org/10.1016/j.molcell.2017.11.015, the disclosures of which are incorporated herein by reference. In some embodiments, an “enrichment” or amplification for promoter sequences, or for specific set of TF binding sites, may be performed on the sequencing library prior to the sequencing step. The enrichment for sequences may be performed by any method that would be appreciated by one of skill in the art. For example, enrichment may be performed using commercially available target capture kits, such as myBaits hybridization capture kits from Arbor Biosciences.

Accordingly, in some embodiments of the inventive concept, “subnucleosome enrichment” may include an enrichment of cfDNA fragments, e.g., cfDNA fragments between about 40-50 bp and about 100 bp or about 40-50 bp and about 147 bp, for example, less than about 147 bp, less than about 100 bp, less than about 90 bp, less than about 80 bp, or even less than about 50 bp, such as cfDNA fragments associated with subnucleosomes, transcription start sites (TSS) and/or TF binding sites for transcriptionally active genes, for example, fragments about 125 bp, about 103 bp, or about 90 bp in size, which have a size less than cfDNA fragments typically associated with nucleosomes and/or chromatosomes, i.e., cfDNA fragments of about 160 bp, for example, about 155 bp to about 170 bp.

It will be appreciated that cfDNA fragments shorter than about 147 bp, fragments associated with subnucleosomes, arise during transcription. Presence of, or an “enrichment” of, these short fragments map the location of subnucleosomes and correlates with promoter activity and/or gene expression, whereas transcriptionally inactive regions will exhibit cfDNA fragments associated with nucleosomes and/or chromatosomes. Accordingly, the length of cfDNA fragments at promoter-proximal regions can be used to determine the expression state of a gene in the cells of origin of cfDNA, and can be used to generate expression signatures for the cells that shed/generate cfDNA, which can be used to identify disease states. Genes, for example, those included as part of an examination of gene expression state may include genes associated with the TF binding sites mapped and identified as described above. Accordingly, expression states/patterns of the genes associated with the TF binding sites mapped and identified as described above, i.e., through subnucleosome analysis at promoters associated with TF binding sites through cfDNA sequencing and analysis, may provide a “signature” for the cellular origin of the cfDNA fragments. In some embodiments, subnucleosome analysis may include selectively removing DNA fragments greater than about 300 bp, greater than about 250 bp, greater than about 200 bp, greater than about 170 bp, greater than about 160 bp, greater than about 155 bp, greater than about 150, or greater than about 147 bp from analysis.

According to some embodiments of the inventive concept, the maps of subnucleosomes associated with/at promoters, TF binding sites and/or gene expression are provided by sequencing and mapping of cfDNA fragments less than about 147 bp, i.e., fragments shorter than those protected by/associated with nucleosomes, e.g., less than about 100 bp, less than about 90 bp, less than about 80 bp, or less than about 50 bp, for example between about 40-50 bp and about 100 bp, i.e., cfDNA fragments typically associated with subnucleosomes, to a number of genes in the genome. Expressed genes exhibit a higher frequency of these cfDNA fragments, subnucleosomal fragments, and a lower frequency of cfDNA fragments of about 160 bp, for example, about 155 bp to about 170 bp, i.e., nucleosomal fragments, when compared to non-expressed genes. Accordingly, in some embodiments, subnucleosomes associated with/at promoters, TF binding sites and/or gene expression are mapped by an increased presence of subnucleosomal cfDNA fragments, over that shown in non-expressed genes. In some embodiments, methods of the present inventive concept can reduce the sequencing information required, and associated cost and resources used in sequencing, according to conventional methods. For example, methods using fast Fourier transformation (FFT) for mapping TF binding and/or gene expression to analyze cell types contributing to cfDNA (see, Snyder et al. 2016, Cell 164, 57-68) require extracting information regarding periodicity of nucleosomes in the region between the transcription start site (TSS) and the TSS+5,000 bp, i.e., 5,000 bp of sequencing information is required for every TSS/gene that is part of the analysis. According to methods of the present inventive concept, sequence information from the TSS+about 300 bp is required for analyzing cell type contribution to cfDNA and promoter activity for every TSS/gene.

As discussed herein, the maps of subnucleosomes associated with/at promoters, TF binding sites and/or gene expression provided by, for example, methods according to the inventive concept, and “signature” provided therefrom, may be used to identify the cellular origin of the mapped cfDNA. It will be appreciated by one of skill in the art that most cfDNA in healthy individuals is generated by normal turnover of lymphoid and myeloid tissue. Accordingly, cfDNA from a subject who is free of a disease or disorder, such a subject that does not have the disease or disorder, or has been successfully treated for the disease or disorder, or monitoring efficacy or progress of treatment for a disease or disorder may be expected to exhibit a maps of subnucleosomes associated with promoters, TF binding sites and/or gene expression shown for, matching, or corresponding to, a signature for lymphoid and myeloid tissue/cells. In contrast, cfDNA from a subject suffering from a disease or disorder, or suffering from relapse of a disease or disorder following treatment, may exhibit a map of subnucleosomes associated with promoters, TF binding and/or gene expression matching, associated with, or corresponding to, a signature for the disease or disorder, including providing information regarding the cellular origin of the disease or disorder. Mapping of transcription factor-nucleosome dynamics from plasma cfDNA is discussed in Rao et al. (2021) doi.org/10.1101/2021.04.14.439883, the disclosures of which are incorporated herein by reference.

In some embodiments, the signature for presence of a disease or disorder may be provided by mapping subnucleosomes associated with/at promoters, TF binding sites, and/or gene expression in cells associated with a disease or disorder, for example, cancer cells. In some embodiments, the cells associated with a disease or disorder from which the signature is provided may be cells from a patient-derived xenograft (PDX) from cancer cells. The cancer, and cancer cells derived therefrom, including PDXs from cancer cells, is not particularly limited. Exemplary cancers include, for example, breast cancer, liver cancer, kidney cancer, pancreatic cancer, thyroid cancer, lung cancer, esophageal cancer, head and neck cancer, colon cancer, rectal cancer, colorectal cancer, gastric cancer, intestinal cancer, gastrointestinal cancer, cervical cancer, uterine cancer, ovarian cancer, bladder cancer, prostate cancer, skin cancer, brain cancer, and/or any metastases of any thereof. In some embodiments, the cancer may be one for which there is a need for improved methods of screening and/or detection, e.g., lung cancer, ovarian cancer, and pancreatic cancer, and/or any metastases thereof. In some embodiments, the cancer cells may be from a breast cancer, such as an ER⁺ breast cancer, a prostate cancer, or a lung cancer, such as a non-small cell lung cancer (NSCLC) or, in some embodiments, the cells may be from a PDX derived from a cancer or cancer cells as described herein.

Transcription factors (TFs) that may be used/analyzed in the methods of the present inventive concept, e.g., for mapping subnucleosomes associated with promoters, TF binding sites and/or gene expression, are also not particularly limited, and may be any TF that may be associated with a disease state or indicative of absence of disease. In some embodiments, the TF used/analyzed may include PU.1. In some embodiments, the TF used/analyzed may include EGR2. In some embodiments, the TF used/analyzed may include CCCTC-binding factor (CTCF). In some embodiments, the TF used/analyzed may include FOXA1. In some embodiments, the TF used/analyzed may include the estrogen receptor (ER).

Similarly, analysis of genes, and expression thereof, by the method of the present inventive concept, may include any gene or genes that may be associated with a disease state, for example, a cancer, or indicative of absence of disease. In some embodiments, for example, genes and gene expression associated with ER and/or FOXA1 binding may be analyzed to provide information regarding ER-positive breast cancer, for example, indication of the presence of, absence of, and/or recurrence of ER-positive breast cancer. In some embodiments, the genes included in the analysis may include genes without other genes overlapping within (±) about 300 bp, about 500 bp, about 1,000 bp about 2,000 bp, or about 5,000 bp from the transcription start site (TSS). In some embodiments, the genes may include the genes (about 13,000) as set forth in the large table entitled 151077-00034_Gene_List.txt, filed Sep. 17, 2020 via EFS-Web with U.S. Provisional Application Ser. No. 63/079,589, the disclosure of which is incorporated by reference in its entirety, or any subset thereof. The total number of genes included for the analysis is not particularly limited, for example, the number of genes may be any number between about 5,000 and about 200,000, e.g., ˜13,000, ˜25,000, ˜40,000, ˜50,000, ˜100,000, or ˜141,000, however, it will be appreciated that including fewer genes in the analysis, in addition to reducing the extent of sequencing performed for each gene, will reduce time/labor/cost of/involved with the analysis. The location of sites in an analysis of ER binding in MCF7 cells are listed in the large table entitled MCF7_ER_bed.txt, the location of sites in an analysis of FOXA1 binding in MCF7 cells are listed in the large table entitled MCF7 _FOXA1_bed.txt, and the location of sites in an analysis of ER binding in UCD12 cells are listed in the large table entitled UCD12_ER_bed.txt, filed Sep. 17, 2020 via EFS-Web with U.S. Provisional Application Ser. No. 63/079,589, the disclosures of each of which are incorporated by reference in its entirety.

Without wishing to be bound by any particular theory, diseases and disorders that may be followed and/or monitored by embodiments of the inventive concept include, for example, cancers, such as, but not limited to, breast cancer, liver cancer, kidney cancer, pancreatic cancer, thyroid cancer, lung cancer, esophageal cancer, head and neck cancer, colon cancer, rectal cancer, colorectal cancer, gastric cancer, intestinal cancer, gastrointestinal cancer, cervical cancer, uterine cancer, ovarian cancer, bladder cancer, prostate cancer, skin cancer, brain cancer, and any metastases of any thereof. In some embodiments, the cancer may be one for which there is a need for improved methods of screening and/or detection, e.g., lung cancer, ovarian cancer, and pancreatic cancer, and/or any metastases thereof. In some embodiments, the cancer may be breast cancer, such as ER⁺ breast cancer, prostate cancer, or lung cancer, such as NSCLC. In other embodiments, the disease or disorder followed and/or monitored may include systemic inflammatory states, such as in, for example, inflammatory bowel disease, systemic lupus or response to immune therapy. Systemic inflammatory states may be monitored based on immune footprints of cfDNA from lymphocytes, monocytes/macrophages and NK cells. Analysis of cfDNA may also be used to monitor TFs and TF binding associated with and specific to disease states, such as EGR2 for M1 versus M2 state of macrophage differentiation, in combination with cell specific gene expression profiles inferred through cfDNA analysis. In other embodiments, analysis of cfDNA can be used for real time disease monitoring during therapy to help determine the extent of disease and distinguish response versus disease progression. In still other embodiments, analysis of cfDNA can be used to individualize care and patient selection based on accurate definition of specific disease states, and to switch therapy when appropriate. Still other embodiments of the inventive concept include predicting treatment outcome, for example, treatment outcome of cancer, such as treatment of NSCLC with an immunotherapeutic, such as pembrolizumab.

Having described various aspects of the present inventive concept, the same will be explained in further detail in the following examples, which are included herein for illustration purposes only, and which are not intended to be limiting to the inventive concept.

### EXAMPLE 1

### Transcription Factor Binding Signatures in Cell-Free DNA (cfDNA) to Detect Disease and Track Treatment Response

### Methods

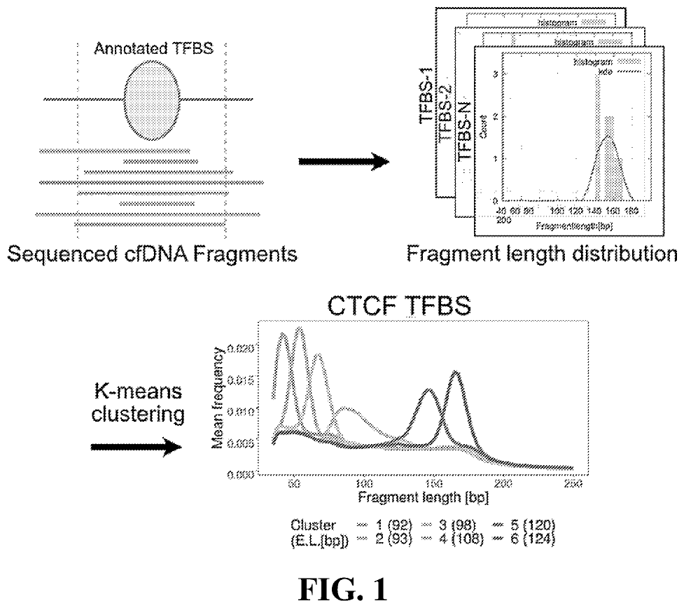

We extract cfDNA from i) 250-500 μl of mouse plasma, and ii) 1-2 ml of human plasma. We extract DNA from plasma using commercially available kits and make sequencing libraries for paired-end Illumina sequencing. Since cfDNA is highly nicked, shorter fragments, which are most important for our analyses, are lost during standard library preparation. Hence, we prepare sequencing libraries from cfDNA that have been denatured into single stranded DNA—Single Strand library Protocol (SSP, Snyder et al., 2016, Cell 164, 57-68). These libraries are subjected to paired-end sequencing in Illumina sequencers. Paired-end sequencing enables us to infer both location of fragment in the genome and the length of the fragment. We then use a reference set of transcription factor binding sites (TFBS) either publicly available (ChIP-seq datasets) or generated in our labs (CUT&RUN) datasets and determine the fragment size distributions at these putative TFBS. We then cluster the TFBS based on the fragment size distribution using k-means method and determine the expected fragment size for each cluster (FIG. 1).

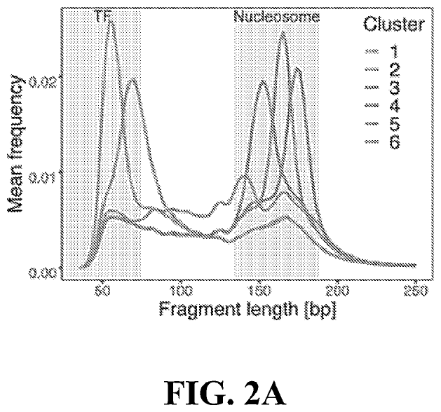

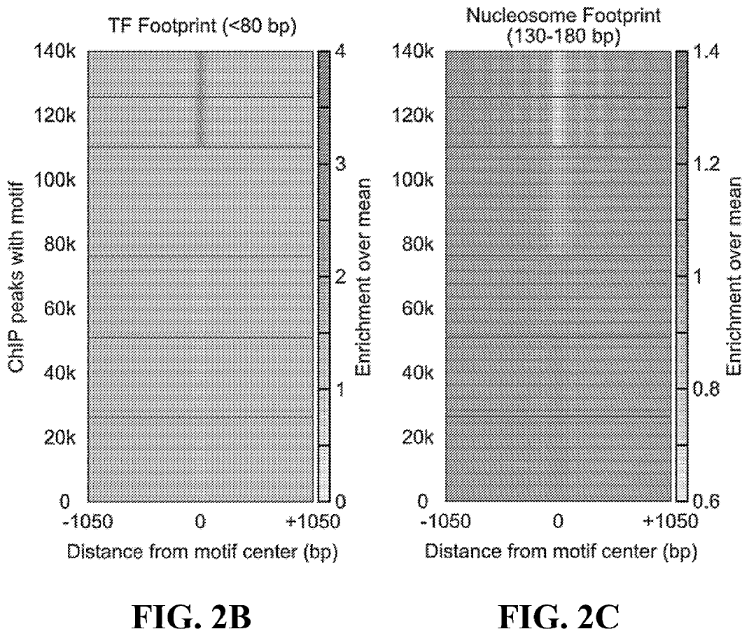

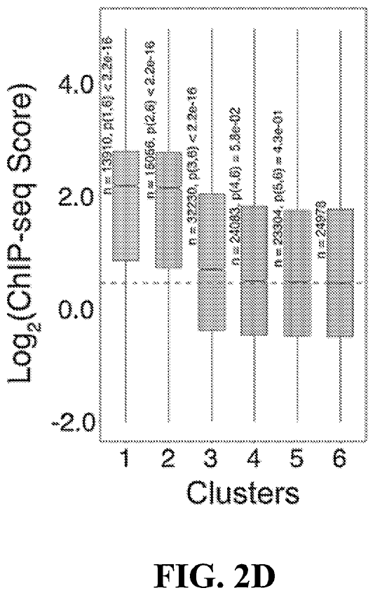

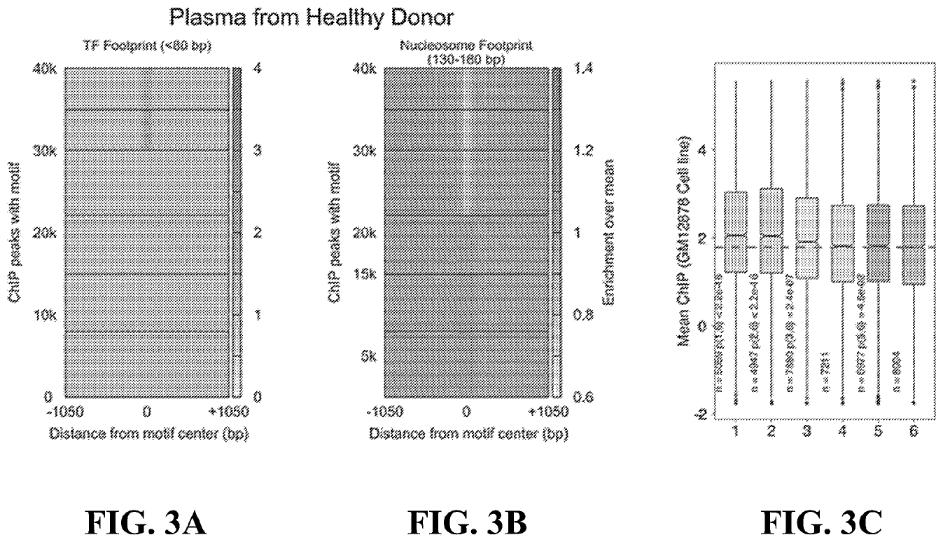

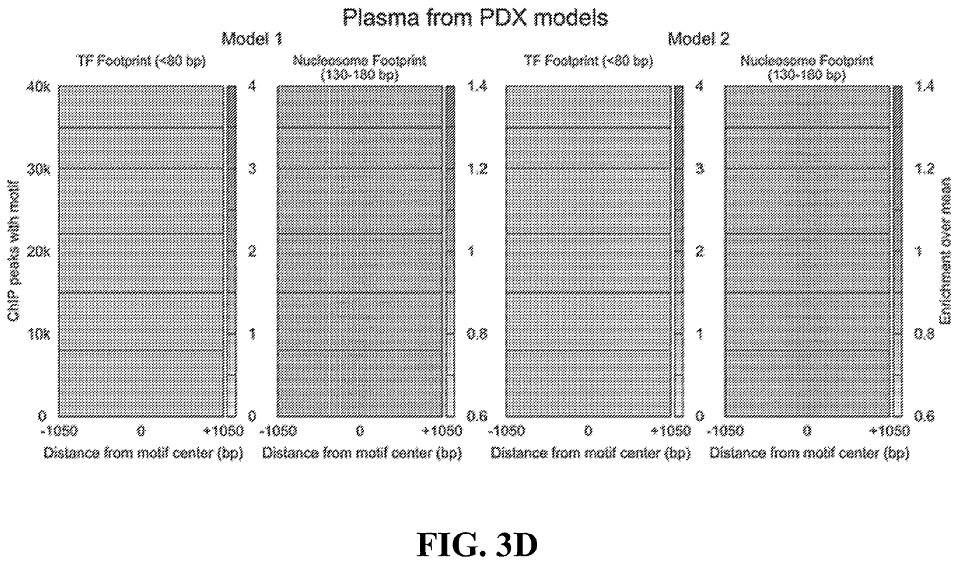

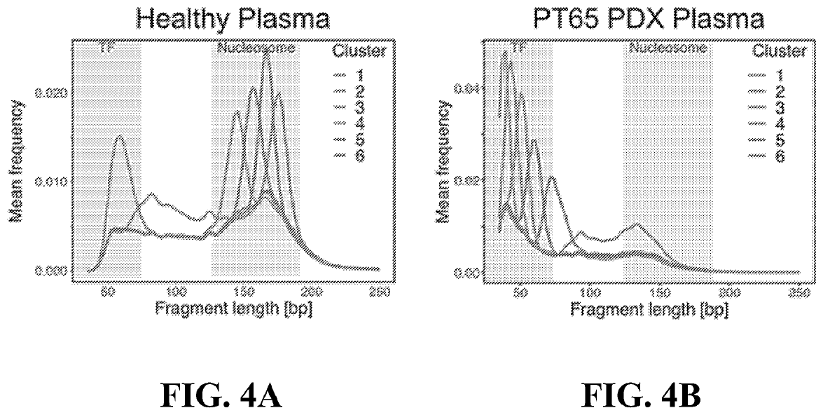

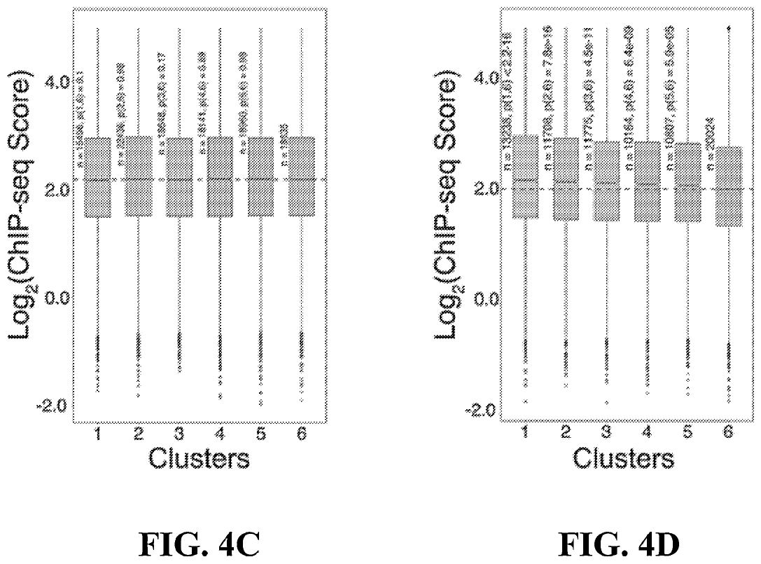

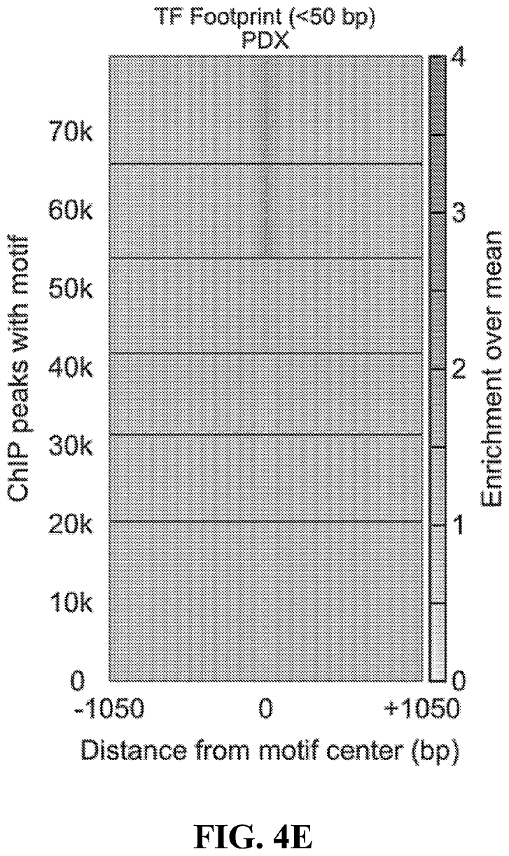

We order the clusters based on their expected fragment size. The clusters with lowest fragment sizes correspond to TFBS that show TF binding in vivo, in the tissue of origin. As a general principal of this approach we show results for three different TF classes:
1) Constitutive TFs (CTCF). We clustered ˜141,000 binding sites of CCCTC-binding factor (CTCF) based on the fragment size distribution at each site and obtained 6 clusters. Strikingly, the clusters separated based on either featuring predominantly short fragments (clusters 1 and 2, FIG. 2A) or featuring nucleosomal fragments (clusters 3-6, FIG. 2A). Thus, we were able to identify sites enriched for TF footprints that featured depleted nucleosomes (FIG. 2B, 2C). The subset of sites that featured nucleosome depletion displayed strong nucleosome phasing upstream and downstream of the TFBS, a well-known feature of active CTCF binding seen in cells in vitro (FIG. 2C). Our observation of enrichment of short footprints at TFBS, depletion of nucleosomes at TFBS, and strong phasing of nucleosomes adjacent to TFBS, strongly suggests that we can simultaneously map nucleosomes and TFs at high resolution from cfDNA prepared using SSP. To further confirm that the identified footprints represent TF binding in tissue of origin, we compared the ChIP-seq scores from a lymphoblastoid cell line (GM12878), for the different clusters. Remarkably, clusters 1, and 2 that featured predominantly short footprints had significantly higher ChIP-seq scores compared to other clusters with nucleosomal footprints, which further confirms that we can track TF binding at tissues-of-origin (FIG. 2D);
2) Hematopoiesis-specific TFs (PU.1). We clustered ˜40,000 TFBS of a pioneer factor involved in myeloid and B-cell lymphoid development, PU.1 into 6 clusters (FIGS. 3A-3D). The top two clusters based on expected fragment length featured strong protections corresponding to TF-binding, which was also reflected in the strongly positioned nucleosomes around the TFBS for these two clusters. The expected fragment length of the clusters correlated with the ChIP-scores of the TFBS-clusters as determined in GM12878 cells. Thus, our method can track binding of hematopoietic-TFs in healthy individuals. We then plotted cfDNA sequencing data at the same TFBS from PDX models of breast cancer. As the tumor-derived cfDNA in PDX would map to the human genome, and the endogenous cfDNA from the mouse would map to the mouse genome, we identified cfDNA molecules from sequencing that were purely from the tumor and separated them from the host. Breast tumors do not have PU.1 expression, and we see a complete loss of both short protections at the TFBS and the ordered nucleosome arrays around the TFBS at PU.1 binding sites for cfDNA that was purely released by a breast tumor. Thus, PU.1 binding as assayed by our method can detect presence of non-hematopoietic source of cfDNA; and
3) Tumor-specific TFs FOXA1 and Estrogen receptor (ER). We next asked if we could detect tumor-specific TF binding using PDX-derived model of ER+ tumor cells, PT65 in comparison to healthy plasma. Since the tumor-derived cfDNA in PDX would map to the human genome, and the endogenous cfDNA from the mouse would map to the mouse genome, we could identify cfDNA molecules from sequencing that were purely from the tumor. First, we observed only one cluster in healthy cfDNA that corresponded to short footprints, which did not have significantly higher ChIP scores compared to other clusters (FIG. 4A, 4C). Second, we observed completely different length distributions for PDX clusters at FOXA1 sites (FIG. 4B, 4E). The much shorter protections in PDX compared to healthy plasma suggests that we are capturing cancer-specific FOXA1 binding in PDX cfDNA. Furthermore, the clusters with shortest protections had significantly higher ChIP scores compared to cluster 6, which had predominantly nucleosomal footprints (FIG. 4D). In summary, we have the capability to track TF binding in tissues-of-origin from cfDNA from healthy plasma as well as from PDX samples.

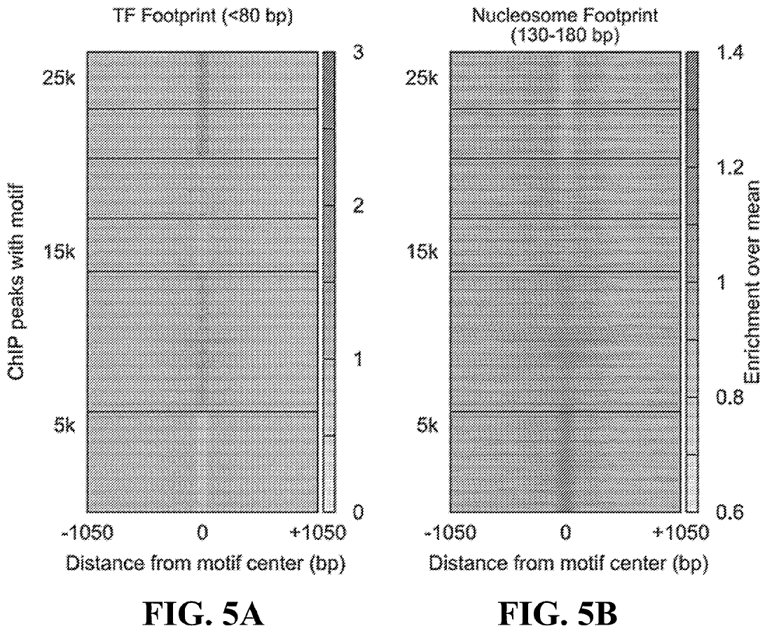

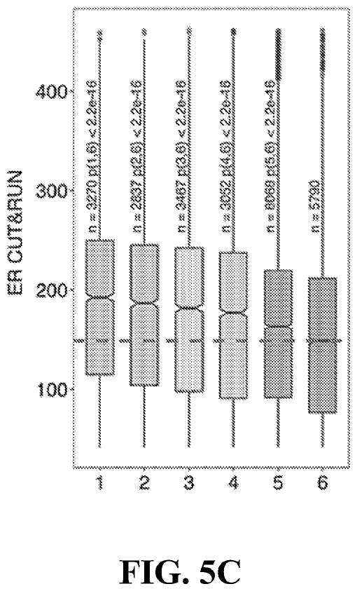

We performed CUT&RUN for ER in MCF7 cells. CUT&RUN is an alternative to ChIP-seq that relies on a protein-A-tagged nuclease that binds to a primary antibody of epitope of choice (here ER). The nuclease is activated upon addition of calcium, which results in release of DNA fragments bound to ER. We obtained ˜25,000 CUT&RUN sites for ER that had sufficient coverage in our PDX data. We performed the same fragment-length analysis at ER TFBS and obtained 6 clusters, where 4 of the clusters with lowest expected fragment length had significantly higher ER binding in vitro and displayed distinct nucleosomal footprints (FIGS. 5A-5C). Thus, defining binding sites in tumor cells using CUT&RUN leads to sensitive mapping of TF-binding in plasma that occurred in tumor-cells-of-origin.

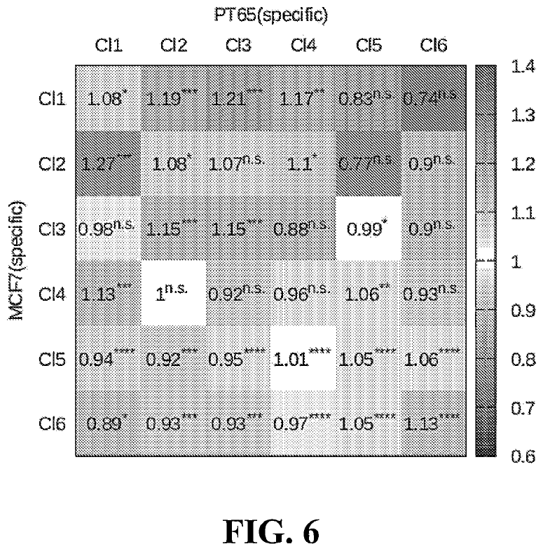

ER is also active in the hematopoietic system and it is important to separate ER-binding in hematopoietic cells from ER-binding in the tumor. To achieve this, we selected the subset of ER CUT&RUN sites that did not feature ER binding in healthy plasma. After removing TFBS that show binding in the hematopoietic system also, we compared the enrichment of sites across different clusters between two PDX models: MCF7 and PT65, which are distinct disease states. We plotted the ratio of observed number of sites overlapping in any two pairs of TFBS clusters (in MCF7 and PT65) to the expected overlap based on chance. We observe significant change in clusters identity between MCF7 and PT65, indicating that the selected ER TFBS can distinguish between disease states (FIG. 6).

### Additional Data

The origin of cfDNA can be determined from an accurate map of the promoter nucleosome dynamics of different cells. Nucleosomes are the organizing subunits of chromatin consisting of an octamer of histones that protect 147 bp of DNA. We found that fragments shorter than 147 bp—“subnucleosomes”—represent DNA unwrapping from the histone octamer during nucleosome disassembly or re-assembly that accompany active transcription. These short “subnucleosome” DNA fragments enabled us to identify, define, and in turn predict the gene expression signature of lymphoid/myeloid tissue in cfDNA from healthy donors, and importantly, detect dramatic changes in cfDNA signatures from donors with cancer. Our method uses signatures of promoter-proximal subnucleosomes to detect cancer. Our approach enables more accurate identification of abnormal patterns of gene expression associated with neoplastic transformation by using the comprehensive information available in cfDNA, circumventing the “needle in a haystack” problem of identifying few tumor mutations to define cell origin. Our new subnucleosome method can be used for disease identification, for predicting treatment response, and for non-invasive early detection. Our method can also be used in combination with profiling transcription factor binding in cfDNA to provide additional information on disease state.

### Results

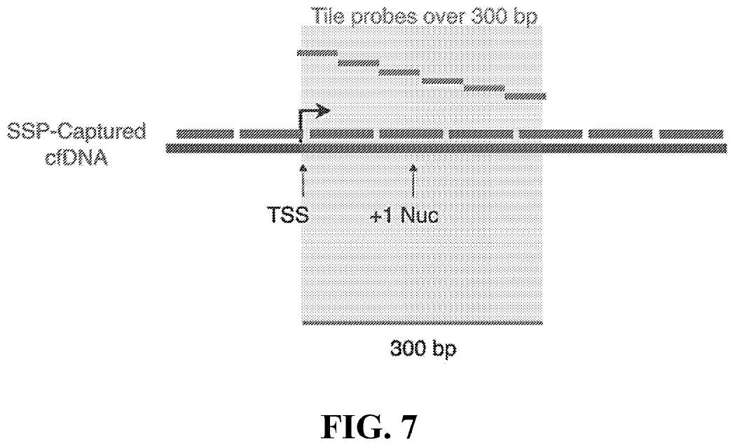

Because subnucleosome enrichment requires information only from 0.15% of the genome, targeted enrichment of promoters using custom oligonucleotides prior to sequencing can dramatically reduce sequencing costs. A custom method to enrich promoter sequences in cfDNA that retains accurate representation while allowing a reduction in sequencing cost is provided. As a demonstration for enrichment of promoter sequences, we performed pooled enrichment of 8 cancer plasma cfDNA samples (Breast and prostate cancer), and 9 healthy plasma cfDNA samples. Commercial tiled oligo probes spanning promoter sequences of about 13,000 genes were designed and obtained, as depicted in FIG. 7.

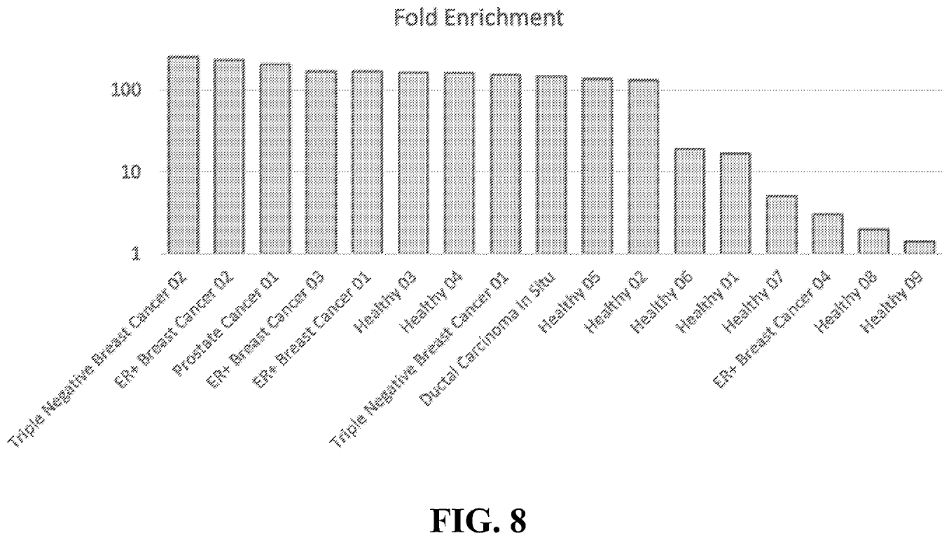

SSP libraries were pooled and then enrichment was performed followed by sequencing. Promoter reads in the enriched libraries were compared to that of unenriched libraries to estimate the extent of enrichment. Enrichment of >100 fold for 11/17 samples and enrichment of >10 fold enrichment for 13/17 samples was obtained, as shown in FIG. 8.

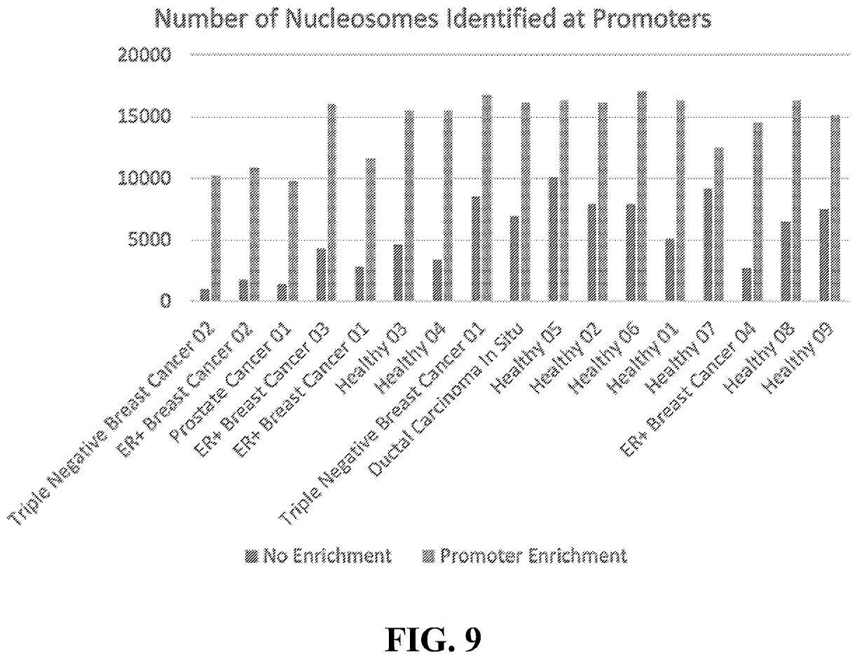

It was then asked if this enrichment in promoter sequences enabled us to identify more +1 nucleosomes. We were able to identify >10,000 promoter nucleosomes in all but one samples, as shown in FIG. 9.

Thus, these experiments show robust enrichment of promoter sequences, which enable sensitive detection of change in gene expression profiles inferred from plasma subnucleosomes in the presence of cancer.

### Prediction of Treatment Outcomes of Immunotherapy for Non-Small Cell Lung Cancer (NSCLC)

Most lung cancer patients are diagnosed in advanced stages where prognosis is dismal, still life-prolonging therapy may increase prognosis by years. Immunotherapy, i.e. immune checkpoint inhibitors (ICI) that block the PD1-PD-L1 axis, have been recently approved and generally implemented for non-curable NSCLC, either as monotherapy (in patients with tumors where >50% of tumor cells express PD-L1) or in combination with chemotherapy. The markers used today to define patients that should be offered ICI, mainly immunohistochemistry (querying PD-L1 levels) are suboptimal: recent studies have shown 20% to 30% of PD-L1-negative patients were responders compared to 50% of PD-L1-positive patients in treatment of metastatic melanoma. Thus, patients denied therapy could in fact have a long-lasting effect, and a substantial fraction of patients that are offered therapy today are not demonstrating a benefit. Since subnucleosome dynamics at the promoter reflects composite gene activity of the tumor and the immune system, it can be used as a signature for overall disease state. Thus, subnucleosome enrichment determined from cfDNA can predict treatment outcomes of immunotherapy in subjects suffering from NSCLC. Finding novel biomarkers for detection of responders as well as early indicators of relapse, are vital for increased survival, and can lead to more optimal usage of limited health resources. Furthermore, uncovering unknown resistance mechanisms can lead to novel treatments. Finally, early implementation of blood-based biomarkers for immunotherapy can reduce treatment costs.

Sequencing of cfDNA is performed on plasma samples from patients who have been treated with pembrolizumab as a first line treatment for metastatic NSCLC. Blood samples are drawn just before the first dose, and 1 day to 1 week before the start of treatment. The treatment duration will vary depending on response. Response is evaluated by CT scans every 8-12 weeks. Samples are from patients with no or minor response (<6 months of treatment), and from patients with prolonged benefit of the medication (>1 year of treatment). Fragment length distributions are obtained genome-wide from the cfDNA sequencing data when determining chromatin protections in cfDNA. Subnucleosome enrichment is calculated at each gene promoter for each sample. Subnucleosome enrichment from patients with good response are compared to the subnucleosome enrichment from patients with poor response by calculating the Log 2 standardized fold-change between the two groups, (μ₁−μ₂)/σ (difference in 2 group means divided by standard deviation in the Log 2 scale). Several genes (117) having standardized fold changes greater than 1.5 have been observed in responders to treatment compared with non-responders to treatment, with the largest standardized fold change being 16. Thus, robust differences in cfDNA subnucleosomes between responders and non-responders to pembrolizumab have been observed in samples collected prior to treatment and indicates that cfDNA signatures can predict treatment response. More importantly, since markers reflect gene activity in the tumor and/or immune system, the cfDNA signatures can inform on mechanisms of treatment resistance in humans.

### Prediction of Treatment Outcomes of Immunotherapy for Melanoma

Similar to prediction of treatment outcomes of immunotherapy for NSCLC, sequencing of cfDNA is performed on plasma samples from patients who have been treated for melanoma using immunotherapy. Samples are drawn from patients with no or minor response, and from patients with prolonged benefit of the medication. Fragment length distributions are obtained genome-wide from the cfDNA sequencing data when determining chromatin protections in cfDNA. Subnucleosome enrichment is calculated at each gene promoter for each sample. Subnucleosome enrichment from patients with good response are compared to the subnucleosome enrichment from patients with poor response by calculating the Log 2 standardized fold-change between the two groups, (μ1−μ2)/σ (difference in 2 group means divided by standard deviation in the Log 2 scale). Genes having standardized fold changes greater than 1.5 are observed in responders to treatment compared with non-responders to treatment. These gene expression differences in cfDNA subnucleosomes between responders and non-responders are used as cfDNA signatures to predict treatment response of immunotherapy for melanoma.

### Predicting Treatment Outcome of Endocrine Therapy for Breast Cancer

Similar to prediction of treatment outcomes of immunotherapy for NSCLC, sequencing of cfDNA is performed on plasma samples from patients who have been treated for breast cancer using endocrine therapy. Samples are drawn from patients with no or minor response, and from patients with prolonged benefit of the medication. Fragment length distributions are obtained genome-wide from the cfDNA sequencing data when determining chromatin protections in cfDNA. Subnucleosome enrichment is calculated at each gene promoter for each sample. Subnucleosome enrichment from patients with good response are compared to the subnucleosome enrichment from patients with poor response by calculating the Log 2 standardized fold-change between the two groups, (μ1−μ2)/σ (difference in 2 group means divided by standard deviation in the Log 2 scale). Genes having standardized fold changes greater than 1.5 are observed in responders to treatment compared with non-responders to treatment. These gene expression differences in cfDNA subnucleosomes between responders and non-responders are used as cfDNA signatures to predict treatment response of endocrine therapy for breast cancer.

### EXAMPLE 2

### Mapping Transcription Factor-Nucleosome Dynamics from Plasma cfDNA

### Introduction

Transcription factors (TFs) are at the apex of gene regulation (1, 2). They usually bind small stretches of DNA in a sequence-specific manner (3, 4). The size of the mammalian genomes is several orders of magnitude greater than the size of TF binding motifs. Hence, there are many more transcription factor binding site (TFBS) sequences that occur by chance compared to functional TFBS (5). Although the question of how TFs discriminate functional binding sites from random motif occurrences is still actively investigated (6-10), at least two mechanisms enable us to connect TF binding to cell state. First, the cell type-specific expression of TFs restricts the pool of motifs recognized in a given cell type. Second, most motifs in the genome are occluded by nucleosomes most of the time (11-15). As a result, the sites in the genome bound by any given TF contribute to the epigenomic signature of a cell type. Furthermore, since functional TF binding drives gene regulation, mapping a TF binding sites in a cell also contributes to an understanding of the regulatory landscape of the cell (16, 17). Methods like Chromatin immunoprecipitation with DNA sequencing (ChIP-seq), chromatin immunoprecipitation, exonuclease digestion and DNA sequencing (ChIP-exo) and Cleavage Under Target & Release Using Nuclease (CUT&RUN) have been used to identify binding sites of human TFs across cell-types (18-21). Here, we show how to leverage this vast knowledge of TF binding in different cell types to map TF footprints in human plasma.

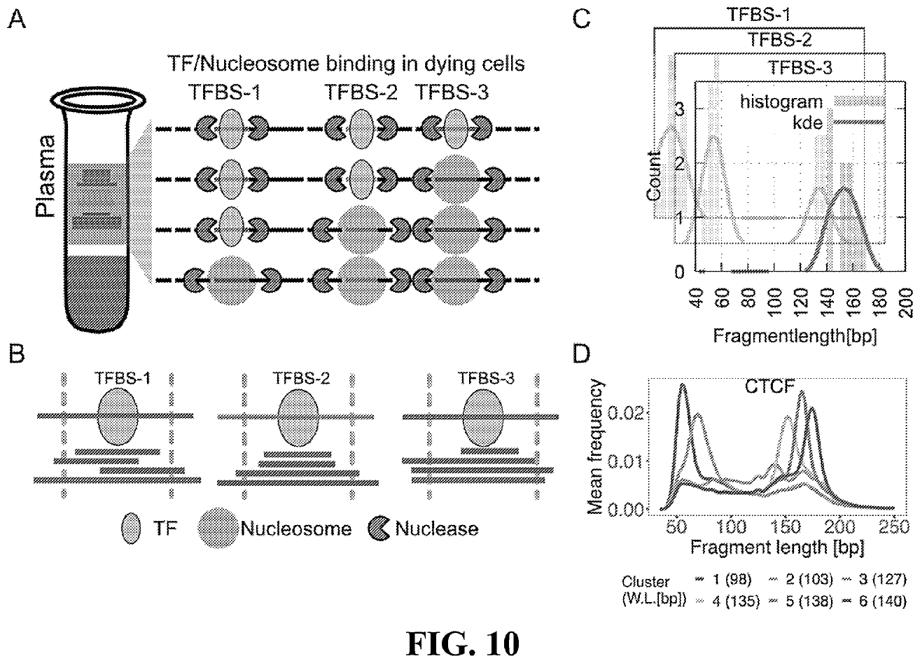

Dying cells in the human body release their content into the bloodstream (22). Genomic DNA that is bound by nucleosomes and TFs escapes endogenous nucleases and so remains protected in plasma (FIG. 10, panel A, (23)). Fragmentomics seeks to uncover tissue-of-origin of cfDNA using the information in cfDNA fragment length. Fragmentomics had its earliest application in prenatal diagnosis and is now being explored as an alternative to mutations and methylation profiling to identify cfDNA tissue-of-origin in cancer (24-26). cfDNA properties such as promoter nucleosome dynamics, locus-specific fragment length distribution, nucleosome-spacing in gene bodies, and nucleosome depletion at promoters have been used to identify tissue-of-origin of cfDNA in order to aid detection of cancer (23, 27, 28). Since TFs and nucleosomes protect distinctly different lengths of DNA, cfDNA facilitates direct mapping of protein-DNA interactions in their cells-of-origin (23). TF binding from cfDNA has also been characterized by averaging across thousands of putative sites, either looking at short protections (23) or by inferring TF binding by nucleosome depletion at TFBS (29).

Regular turnover of lymphoid/myeloid cells in the human body is the major contributor to the pool of cfDNA in plasma (30). However, in the presence of cancer, a detectable fraction of cfDNA also arises from tumors (31, 32). This suggests that cfDNA has the potential to map the tumor epigenome in real-time, and therefore can help uncover the regulatory landscape of cancer from plasma. Here, we map TF footprints in plasma cfDNA by combining library protocols that enrich for short fragments with computational methods that identify the subset of TFBS that leave footprints in plasma. We show that the strength of TF footprints in plasma is proportional to the binding strength of the TF in the tissue-of-origin of the cfDNA fragments, which can enable the mapping of regulatory landscapes of tumors from plasma. As proof of principle, we demonstrate that plasma TF footprints in an estrogen receptor positive (ER+) breast cancer model can predict TF-specific accessibility across human tumors, which raises the possibility of mapping tumor TF binding in human plasma. We then identify TFBS where the density of TF footprints in human plasma samples can be used to identify the presence of breast cancer. ER+ breast cancer is one of many examples of a TF driven disease: the cancer state, that is, response or resistance to drug is reflected by where in the genome ER (a TF) and related TFs like FOXA1 can bind in tumor cells (33-35). Thus, our results show that plasma cfDNA contains TF binding information that is specific to tumor state.

### Materials

### Plasma Samples

Plasma sample information is described in Table 1.

TABLE 1
Plasma samples used in this study.

| Sample | | | |
|---|---|---|---|
| name | Source name | Sex | Disease status |
| MCF7 | Cell line | F | ER+ breast cancer |
| UCD4 | Breast tumor | F | Breast cancer with ER |
| | xenograft | | mutation |
| UCD65 | Breast tumor | F | Breast cancer with ER |
| | xenograft | | amplification |
| F02 | Cell-free DNA | F | Healthy |
| F05 | Cell-free DNA | F | Healthy |
| SporeD3 | Cell-free DNA | M | Healthy |
| BC02 | Cell-free DNA | F | ER+ breast cancer |
| BC03 | Cell-free DNA | F | ER+ breast cancer |
| SporeA2 | Cell-free DNA | M | Lung cancer (non-small) |
| SporeB2 | Cell-free DNA | F | Lung cancer (small cell) |
| SporeF2 | Cell-free DNA | M | Lung cancer (squamous) |
| SporeG2 | Cell-free DNA | M | Lung cancer (Adenocarcinoma) |

### ChIP-seq Peaks

We collected ChIP-peaks from publicly available datasets (18, 63, 64). We obtained clustered peaks for CTCF and PU.1 from ENCODE (http://hgdownload.cse.ucsc.edu/goldenPath/hg19/encodeDCC/wgEncodeRegTfbsClustered/wgEncodeRegTfbsClusteredV3.bed.gz). For LYL1, we used peaks from ReMap (http://remap.univ-amu.fr/storage/remap2020/hg38/MACS2/TF/LYL1/remap2020_LYL1_all_macs2_hg38_v1_0.bed.gz).

### TF Motifs

We used TF motifs from JASPAR (65) (CTCF:

http://jaspar.genereg.net/matrix/MA0139.1/, PU.1: http://jaspar.genereg.net/matrix/MA0080.5, ER: http://jaspar.genereg.net/matrix/MA0112.1; http://jaspar.genereg.net/matrix/MA0112.2; http://jaspar.genereg.net/matrix/MA0112.3, FOXA1: http://jaspar.genereg.net/matrixMA0148.1; http://jaspar.genereg.net/matrix/MA0148.2; http://jaspar.genereg.net/matrixMA0148.3) and HOCOMOCO (66) (LYL1: http://hocomoco.autosome.ru/motif/LYL1_HUMAN.H11MO.0.A).

### Genome-Wide Signal

We used publicly available genome-wide signal files in bigwig format to map ChIP and MNase signal to TF binding sites and their flanks. CTCF:

https://www.encodeproject.org/files/ENCFF578TBN/@@download/ENCFF578TBN.bigWig, PU.1:
https://www.encodeproject.org/files/ENCFF324NQZ/@@download/ENCFF324NQZ.bigWig, LYL1: GEO: GSE63484.

### Methods

### cfDNA Extraction

1-4 mL human plasma or 0.2-0.5 mL of mouse serum were thawed from −80° C. storage. Plasma or serum were spun at max speed (21,000 rcf) at 4° C. for 5-10 mins to pellet any cell debris. Supernatant was transferred to new tubes and cfDNA was extracted using the QIAGEN ccfMinElute kit (cat. 55204) and eluted in 30 μL of nuclease-free water and directly added to the single-stranded DNA library protocol (SSP) or stored at −20° C.

### Single-Stranded DNA Library Protocol (SSP)

The capture of cfDNA fragments from plasma or serum was performed similar to Snyder et al. (23). In brief, 1-10 ng cfDNA was dephosphorylated using FastAP Thermosensitive Alkaline Phosphatase (Thermo Scientific cat. EF0651), denatured, and incubated overnight with CircLigaseII (Lucigen cat. CL9025K) and 0.093-0.125 μM biotinylated CL78 primer (23) at 60° C. with shaking every 5 minutes. Captured cfDNA fragments were denatured and then bound to magnetic streptavidin M-280 beads (Invitrogen cat. 11205D) for 30 minutes at room temperature with nutation. Beads were washed and second-strand synthesis was performed using Bst 2.0 DNA polymerase (NEB cat. M0537) with an increasing temperature gradient 15-31° C. with shaking at 1750 rpm. Beads were washed and a 3′ gap fill was performed using T4 DNA polymerase (Thermo Scientific cat. EL0011) for 30 minutes at room temperature. Beads were washed and a double-stranded adapter was ligated using T4 DNA ligase (Thermo Scientific cat. EP0062) for 2 hours at room temperature with shaking at 1750 rpm. Beads were washed and resuspended in 30 μL 10 mM TET buffer (10 mM Tris-HCl pH 8.0, 1 mM EDTA pH 8.0, 0.05% Tween-20). Beads were denatured at 95° C. for 3 min and cfDNA libraries were collected after immediate magnetic separation.

Quantitative real-time PCR was performed on cfDNA libraries using iTAQ Supermix (Bio-Rad cat. 1725124) and Ct values were used to determine the number of PCR cycles needed to amplify each library. PCR was performed with KAPA HiFi DNA polymerase (Kapa Biosystems cat. KK2502) using barcoded indexing primers for Illumina. Primer dimers were removed from the libraries using AMPure beads (Beckman Coulter cat. A63881). Libraries were eluted in 0.1×TE and concentrations were determined using Qubit. The length distribution of each library was assessed by the Agilent Bioanalyzer using the D1000 or HSD1000 cassette. Libraries were sequenced for 150 cycles in paired-end mode on NovaSeq 6000 system at University of Colorado Cancer Center Genomics Shared Resource.

### Cut&Run

We used an immuno-tethered strategy for profiling the binding of the ERα and FOXA1 transcription factor in human MCF7 breast cancer cells. MCF7 cells were estrogen withdrawn for 72 hours before being plated and then treated with either ethanol (vehicle control) or 10⁻¹⁰ M E2 (estradiol) for 1 hour prior to cell collection. The CUT&RUN method uses an antibody to a specific chromatin epitope to tether Protein A-MNase at chromosomal binding sites within permeabilized cells. The nuclease is activated by the addition of calcium and cleaves DNA around binding sites (19). Cleaved DNA is isolated and subjected to paired-end Illumina sequencing to map the distribution of the chromatin epitope genome-wide. We used a primary antibody to human ERα (ab3575, abcam, Cambridge, MA) and human FOXA1 (ab170933) and protein A-MNase fusion (19) (pA-MNase, a gift from S. Henikoff, Fred Hutchinson Cancer Research Center, Seattle WA). CUT&RUN profiling with 5×10⁵ cells and library amplification with 13 cycles of PCR was performed as described (19). Libraries were sequenced for 10 million paired-end reads on the Illumina NovaSeq 6000 platform at the University of Colorado Denver Cancer Center Genomics Shared Resource. Paired-end reads were mapped to the GRch38 assembly of the human genome using Bowtie2 (67).

### Data and Code Availability

All datasets were aligned to the hg38 version of the human genome. Datasets generated in this study have been deposited in GEO under accession GSE171434 and will be made public upon acceptance. All scripts and pipelines used in this study are available at https://github.com/satyanarayan-rao/tf_nucleosome_dynamics.

### Cut&Run Peaks

To call peaks, we used custom python script (deposited in github). Briefly, we first normalized coverage of <120 bp protected fragments in CUT&RUN data at 10 base pair resolution, and then smoothed the coverage with a Savitzky-Golay filter (68) available as a SciPy (69) method ‘signal.savgol_filter’ with parameters window_length=9, polyorder=1. We determined the cut-off for each dataset by iteratively eliminating outliers and used ‘find_peaks’ method in SciPy to call peaks that were separated by at least 250 bp.

### Aligning Mouse Extracted cfDNA to In Silico Concatenated Genome

The names of chromosomes of human (hg38; GRCh38 assembly) and mouse (mm10: GRCm38 assembly) reference genomes were first prefixed by hg38 and mm10 respectively, and then the fasta files were concatenated together to represent an in silico human+mouse genome. We then aligned C/PDX cfDNA to this concatenated genome using bowtie2 (67) with parameters “--local --very-sensitive-local --no-unal --no-mixed --no-discordant -I 10 -X 700”. We selected for mapped reads and then filtered out reads with secondary alignment from the bam file using the command “samtools view -F 4 <bam file> | grep -v ‘XS:’” (70). This filtering ensured that we did not consider any reads that aligned to both human and mouse genomes. To get human aligned reads we filtered for the hg38 prefix in the reads' chromosome name.

### Defining TFBSs Under ChIP-seq Peaks

We first selected for ChIP-seq peaks that do not overlap with ENCODE profiled blacklisted regions, and we considered all peaks except the ones on chromosome Y. We then used FIMO (71) with parameters “--max-stored-scores 10000000 --oc <output-directory> <motif-file> <fasta-file>” to scan for motifs on sequences underlying ChIP-seq peaks. In case of overlapping peaks in 50 bp span, we keep the motif with higher FIMO score. Final number of motifs under ChIP-peaks used for TFs are tabulated in Table 2.

TABLE 2
Transcription factor ChIP-seq peak counts

| Category | CTCF | PU.1 | PU.2 |
|---|---|---|---|
| Total ChIP peaks | 231,309 | 67,558 | 33,709 |
| Chr1-X | 231,075 | 67,502 | 33,681 |
| After blacklisted region filtering | 230,965 | 67,496 | 33,608 |
| Motif discovery | 215,818 | 71,205 | 14,899 |
| Overlapping motifs in ± 50 bp | 97,229 | 17,216 | 6,853 |
| Non-overlapping motif | 118,589 | 53,989 | 8,046 |

### cfDNA Length Distribution Clustering

Length distribution of mapped cfDNA fragments to a TFBS is estimated by ‘density’ function in R with a smoothing bandwidth (bw) of 3 at 100 equally spaced points (n=100) between 35 to 250 bp. Clustering of estimated cfDNA length distribution at individual sites was performed using ‘kmeans’ function in R with parameters: centers=6, iter.max=250, and nstart=20. A cluster is visually represented by the mean of fragment length distributions of sites in that cluster. Weighted length of each cluster was calculated by multiplying fragment length to its normalized frequency. Clusters 1 to 6 were assigned by ranking the clusters by their weighted length.

### Mapping cfDNA Length Class to TFBS and its Flank

Genome-wide cfDNA read density (bigwig) was generated for short (<80 bp) and nucleosomal sized fragments (130-180 bp). First, a bedgraph (coverage of bases genome-wide; no normalization performed) file was generated using bedtools (72) genomecov utility with command line option “-bga” and then bedgraph file was converted to bigwig using kent tools “bedGraphToBigWig” (73). While creating the bigwig file we considered cfDNA fragment center±30 bp (if fragment is >60 bp). Bigwig is mapped to TFBS±1 Kb using pyBigWig module from deeptools (74) and then enrichment over mean (E.O.M) is calculated. E.O.M is smoothed using Savitzky-Golay filter (68) available as a SciPy (69) method ‘signal.savgol_filter’ with parameters window_length=51, polyorder=3.

### ChIP-Seq Score Calculation Sites in cfDNA Length Clusters

For a TFBS in a given cluster, Log 2 of mean fold enrichment over control was calculated for TFBS±300 bp. pyBigWig module from deeptools (74) was used to map signal from bigwig file to defined genomic regions.

### MNase Signal Mapping to CTCF Sites

MNase data from ENCODE (18) was mapped to CTCF motif center±1 kb. E.O.M and smoothing was performed similar to how it was done for cfDNA length class heatmaps (see Mapping cfDNA length class to TFBS and its flank).

### V Plots

For CTCF sites in cfDNA length clusters 1 and 2, cfDNA fragment centers were mapped to CTCF motif center±500 bp. Total number of cfDNA centers of a given length is plotted against the distance of the fragment centers from the CTCF motif center.

### Cut&Run Score Calculation

CUT&RUN score has been calculated as the read density in regions spanning CUT&RUN peak summit±50 bp.

### Defining Significant Sites and Specific Sites

cfDNA length clusters that have significantly higher binding scores (ChIP scores for CTCF, PU.1 and LYL1; CUT&RUN scores for ER and FOXA1) compared to cluster 6 are considered significant i.e., overall, sites in these clusters have stronger binding strength inferred from TF binding experiments compared to cluster 6. Specific sites are identified by subtracting significant sites of one sample from significant sites from another sample. In the case of disease state detection analysis i.e., healthy vs. cancer, cancer-specific sites (CSS) and healthy-specific sites (HSS) were defined. Cancer-specific sites for ER, for example are defined by subtracting sites in healthy plasma (IH02) (23) significant clusters 1 and 2 from UCD65 clusters 1-4. Similarly, healthy-specific sites for ER are defined by subtracting sites from UCD65 clusters 1-4 from IH02 clusters 1 and 2. In the case of cancer state detection analysis i.e., separating tumor subtypes (UCD65 vs. MCF7, UCD4 vs. UCD65, and UCD4 vs. MCF7) using tumor TF binding sites, tumor-specific sites were defined by a similar approach. We did not observe enrichment at FOXA1 binding sites in UCD4 dataset, thus tumor-specific sites were not defined for FOXA1 in UCD4.

### Dilution Analysis

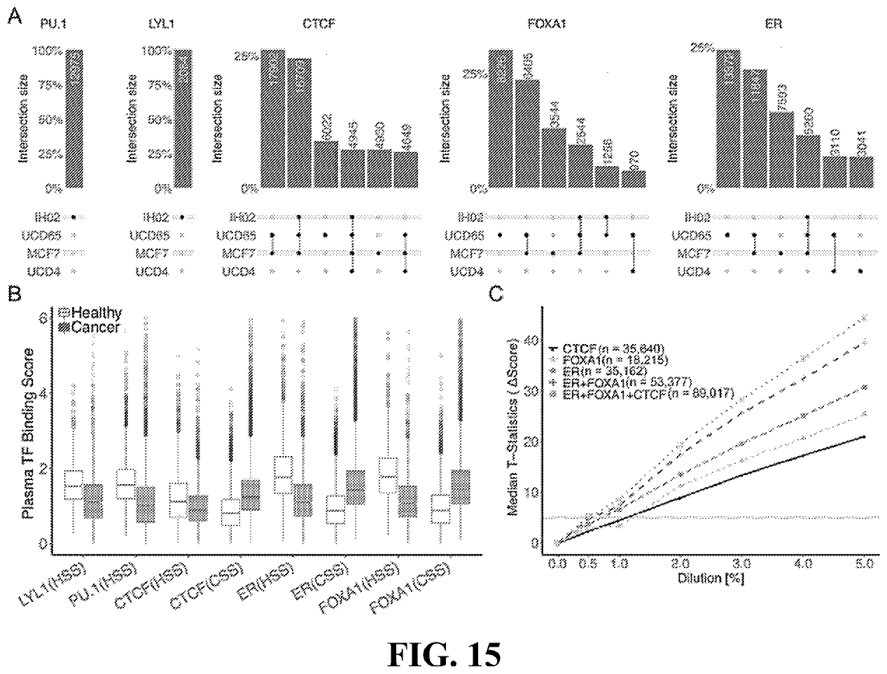

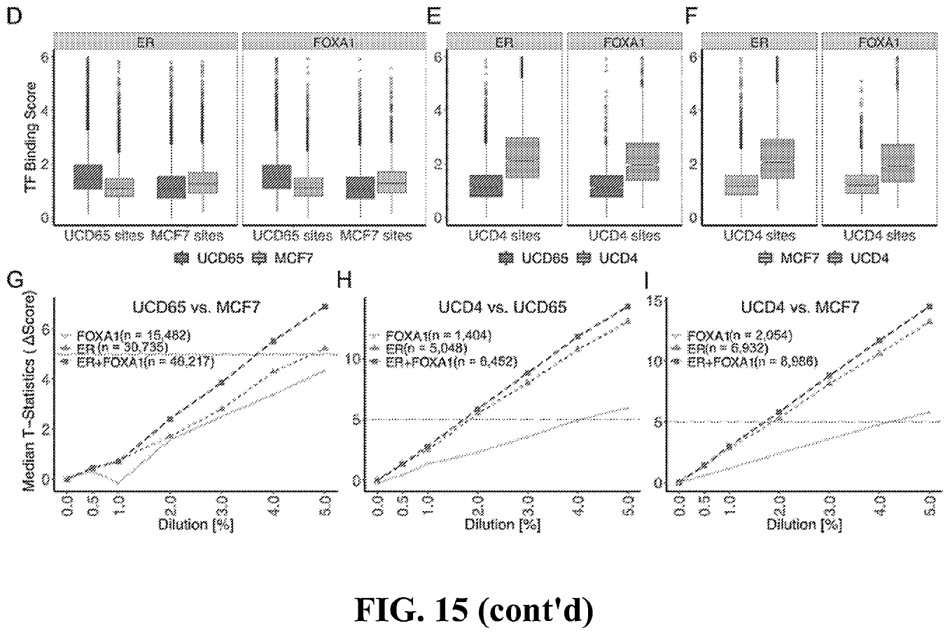

Disease detection. In silico patient data was generated by diluting healthy sample (IH02) (23) with different fractions of UCD65 cfDNA. For each dilution level, 100 in silico patient datasets were generated by randomly sampling reads from IH02 and UCD65 datasets at the ratio defined by the dilution level. For a given cancer/healthy-specific binding site, the TF binding score was calculated as the ratio of the short fragment coverage in (<80 bp) TFBS±50 to the coverage in TFBS±1 kb. Reference TF binding score is calculated just in healthy state, and for each in silico patient dataset, scores are calculated in same fashion. ΔScore (used in FIG. 15, panel C) for cancer specific sites was calculated as the difference between patient and healthy states (gain in score), but for healthy-specific sites the sign was reversed (loss in score). T-test was performed on ΔScore values from all sites (healthy-specific+cancer-specific) to reflect how many standard deviations away the scores are from the healthy reference.

Cancer state detection. For each xenograft (UCD4, UCD65 and MCF7) model, 100 in silco patient data was generated by diluting healthy plasma (IH02) with different fractions of ctDNA. For each of three comparisons of xenograft models, the following were calculated (using UCD65 vs. MCF7 as an example): i) TF binding scores at tumor subtype specific sites using UCD65 and MCF7 in silico patient data respectively, ii) calculated ΔScore for UCD65-specific sites by subtracting scores of MCF7 dilution from UCD65 dilution. Similarly, ΔScore for MCF7-specific sites were calculated by subtracting scores pr UCD65 dilution from MCF7 dilution, and iii) calculated T-statistics on ΔScore using ‘ttest_1samp’ function from scipy.stats module (69) with expected value in null hypothesis=0.

### TCGA ATAC-Seq and Expression Analysis

FPKM files for each cohort were downloaded from TCGA website. FPKM for a gene was converted to TPM using the following formulae:

$$TPM ( {Gene}_{i} ) = \frac{FPKM ( {Gene}_{i} )}{{\sum }_{i = 1}^{N} FPKM ( {Gene}_{i} )} * 1 {0}^{6}$$

where N is the total number of genes found in the FPKM table.
ATAC insert bigwig files from Corces MR et al., (59) were used to map ATAC signal around TF sites (peak±150 bp).

### Cancer vs. Healthy and Breast Cancer vs. Non-Breast Cancer Prediction Analysis

Healthy-specific sites (HSS) and Cancer-specific sites (CSS) were ordered by their binding strength inferred from ChIP (motif center±300 bp; for PU.1, LYL1, and CTCF) or CUT&RUN (summit±100 bp; for ER and FOXA1) and grouped in a bin of size 250 to define TF features. cfDNA-inferred binding score at TF features is defined by the following formulae:

$$Binding Score {( feature )}_{sample} = \frac{{\sum }_{i = 1}^{250} \# short cf {DNA}_{s m a p l e} fragments i n {Site}_{i} \pm 50 bp}{{\sum }_{i = 1}^{250} \# short {cfDNA}_{s m a p l e} fragments i n {Site}_{i} \pm 1 kb}$$

To identify what TF features are class-specific (for example, class1—cancer, class2—healthy), we defined a Z-score metric using the following formula:

$${Z}_{feature} = \frac{{{Mean {( Binding score}_{feature} )}_{class 1} - Mean {( Binding score}_{feature} )}_{class 2}}{[ {S D ( Binding {score}_{feature} )}_{c l a s s 1} + {SD ( Binding {score}_{feature} )}_{class 2} ] / 2}$$

Where SD stands for standard deviation. Features with |Z_{feature}|>1 were selected and depending on the sign were annotated as class1-specific (+ve) or class2-specific (−ve). Enrichment of a TF in particular category (for example healthy-specific) was calculated by abundance of the TF features as Log 2 (Observed frequency/expected frequency).

To predict a class (breast cancer or non-breast cancer) for a cfDNA sample, leave-one-out cross validation approach was adopted where cfDNA sample of our interest was kept away during feature selection process described above. Each sample was then assigned a single score by subtracting the sum of binding scores of features with negative Z-scores (Z_{feature}<-1) from the mean of features with positive Z-scores (Z_{feature}>1) and then dividing by the total number of features (|Z_{feature}>1|). For the left-out sample, distances from the median of two classes were calculated and assigned the class label with closest distance.

### Results

### Unique cfDNA Fragment Length Distributions Identify TF Binding in the Tissue-of-Origin

ChIP-seq and CUT&RUN applied to cell lines and tissue samples represent gold standard methods of determining TF binding across the genome. To study human disease, it is impractical and nearly impossible to perform repeat analyses on biopsy tissues. We therefore set out to develop an alternative to ChIP-seq and CUT&RUN that can be applied to physiological and pathological states of humans in a minimally invasive manner by inferring specific TF binding from plasma cfDNA. TF footprints (<80 bp) are too short to be captured by standard library protocols, but single strand library protocol (SSP) for cfDNA can robustly capture short as well as longer, nucleosomal cfDNA fragments (23). In all our analyses, we used cfDNA sequencing datasets generated using SSP in this study as well as from a published study (23).

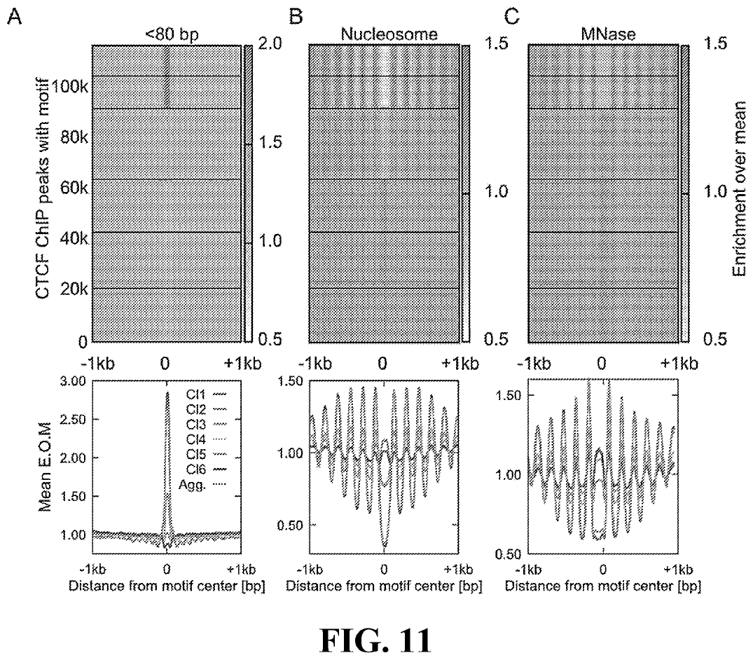

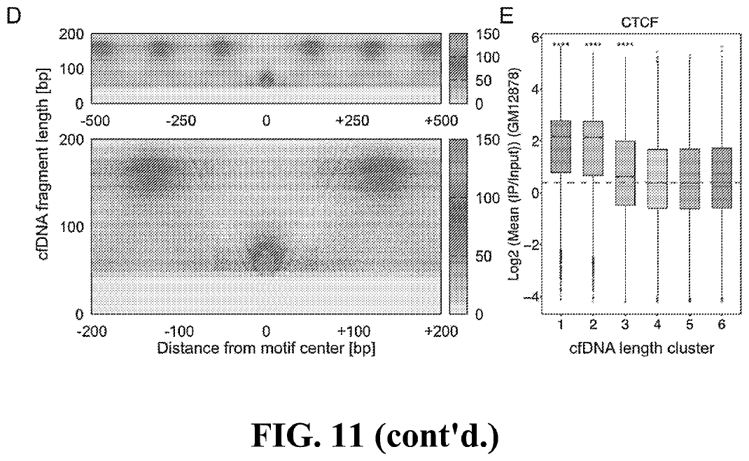

To ask if we can uncover TF-nucleosome dynamics from plasma cfDNA, we undertook a candidate approach of examining binding sites of specific TFs. We started with CTCF as it is constitutively expressed (36, 37), has a long residence time on DNA (38), and has known binding profiles in a large, diverse set of cell types (18). We aggregated CTCF binding sites from 18 cell types (70 cell lines) and analyzed fragment length distributions of cfDNA from a healthy donor (IH02 dataset (23)) at these sites. At each TFBS, we mapped cfDNA fragment midpoints (FIG. 10, panel B) and estimated a fragment length distribution (FIG. 10, panel C). K-means clustering of these fragment length distributions identified two types of clusters—one enriched with short cfDNA fragments (<100 bp; cluster 1 and 2) and the other enriched with long cfDNA fragments (>120 bp; cluster 3-6) (FIG. 10, panel D). When we mapped enrichment of cfDNA fragments around 1 kb of the TFBS, clusters 1 and 2 showed strong enrichment of short protections at TFBSs relative to 1 kb upstream and downstream of the TFBS (FIG. 11, panel A). Strikingly, these two clusters also showed strong nucleosome phasing at least 1 kb upstream and downstream of the TFBS (FIG. 11, panel B). It is well known that CTCF binding organizes nucleosomes in its vicinity (39, 40). Thus, fragment length profile at CTCF binding sites not only identified TF binding, but also uncovered chromatin structure surrounding the bound CTCF from plasma cfDNA. Since most cfDNA in a healthy donor arises from lymphoid/myeloid cells, we asked if the TFBS clustering based on cfDNA reflected nucleosome positioning in a representative lymphoblastoid cell line (GM12878). MNase-seq data (18) from GM12878 showed strong nucleosome phasing for clusters 1 and 2, but the rest of the clusters had very weak or no phasing patterns (FIG. 11, panel C). This strongly suggests that we can capture CTCF binding and associated nucleosome landscape from lymphoid/myeloid cells in cfDNA and that the mechanism of DNA release from these cell types gives a signal similar to MNase profiling.

To further visualize the chromatin structure around CTCF bound sites and identify the minimum protection conferred by CTCF on DNA, we plotted the count of cfDNA fragment midpoints around CTCF bound sites as V-plots for sites in clusters 1 and 2 (41). With the V-plot spanning TFBS±500 bp, we observe strongly positioned nucleosomes with protection length between 140-180 bp, flanking short protections at the CTCF sites in the center (FIG. 11, panel D, top). In the V-plot spanning TFBS±200 bp, a strong “V” is evident at the center, where there is an enrichment of fragments<80 bp. A “V” indicates a well-positioned, strong barrier to nucleases, which further confirms that cfDNA is directly mapping TF binding and its associated nucleosome landscapes from the cells of origin (FIG. 11, panel D, bottom).

The separation of bound and unbound sites by our clustering approach is also apparent when we compare the short and nucleosomal fragment enrichment at individual clusters to the aggregate enrichments across all sites (gray lines in FIG. 11, panel A, bottom). TF enrichment, nucleosome occlusion, and nucleosome ordering are substantially weaker in aggregate compared to clusters 1 and 2 as expected. In other words, identifying the subset of sites that are bound could inform us of TF binding strength in cfDNA cells of origin. To test this idea, we calculated the ChIP scores from GM12878 cells at TFBS belonging to each cfDNA length cluster. We found the ChIP scores of the first two clusters to be almost four times higher than the other four clusters (FIG. 11, panel E). The fact that hematopoietic ChIP scores correlate with our inferred sites of CTCF binding in cfDNA supports the conclusion that cfDNA length profile at TFBS reports on TF binding strength in cfDNA tissue-of-origin.

### Binding Sites of Hematopoietic TFs are Sensitive to Changes in cfDNA Tissues-of-Origin

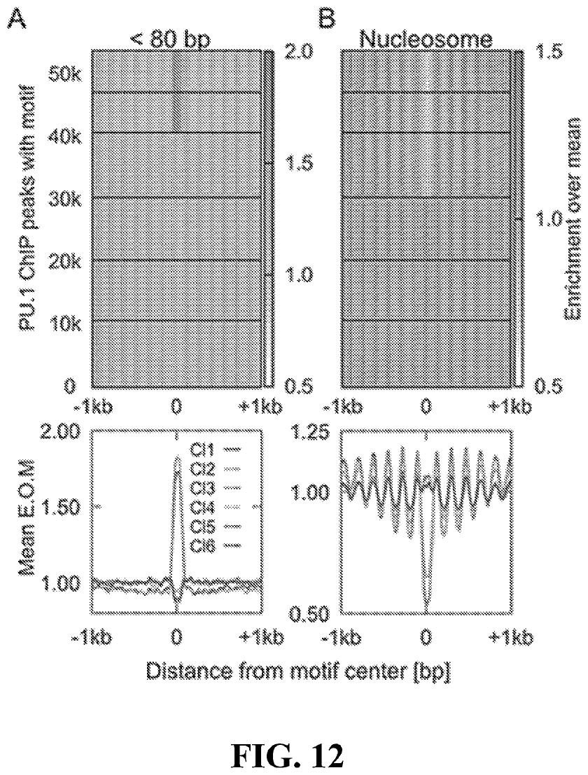

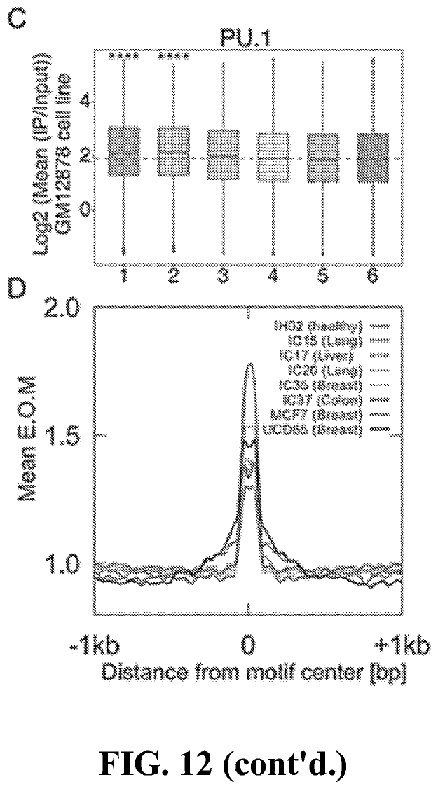

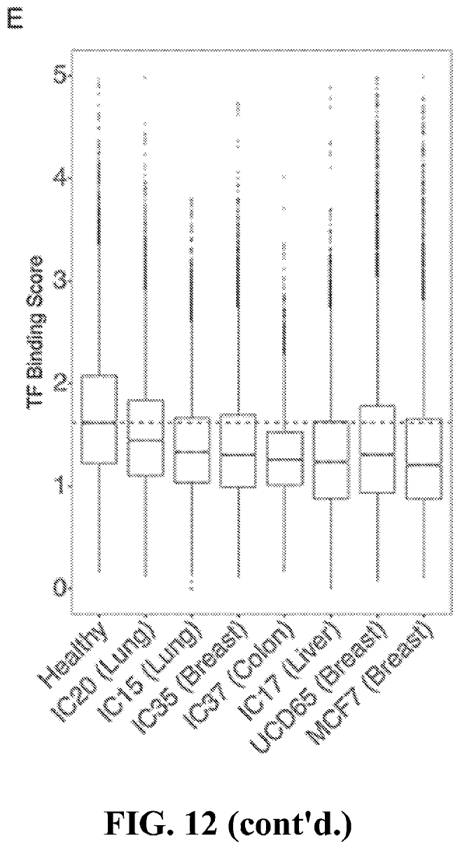

Since most cfDNA in healthy individuals is of lymphoid/myeloid origin, we asked if we can map protections for lymphoid/myeloid-specific TFs: PU.1, a pioneer factor that plays a crucial role in myeloid and B-cell development (42, 43) and LYL1, an important factor for erythropoiesis (44) and development of other hematopoietic cell types (45, 46). Upon clustering the binding sites of PU.1 and LYL1 based on cfDNA length distributions, we found an enrichment of short protections at a subset of binding sites similar to CTCF (clusters 1 and 2; FIG. 12, panels A, F). Distribution of longer fragments around the binding sites showed strong nucleosomal phasing in clusters 1 and 2 (FIG. 12, panels B, G). The presence of nucleosome phasing further confirmed specific TF binding as this is a known outcome of LYL1 and PU.1 binding to DNA (29, 47-49). Clusters 1 and 2, which had the highest enrichment of short protections also had significantly higher ChIP scores in lymphoid/myeloid cells-lines compared to cluster 6 (nucleosomal) for both PU.1 and LYL1 (FIG. 12, panels C, H). Thus, we can map binding of hematopoietic TFs in plasma cfDNA in humans.

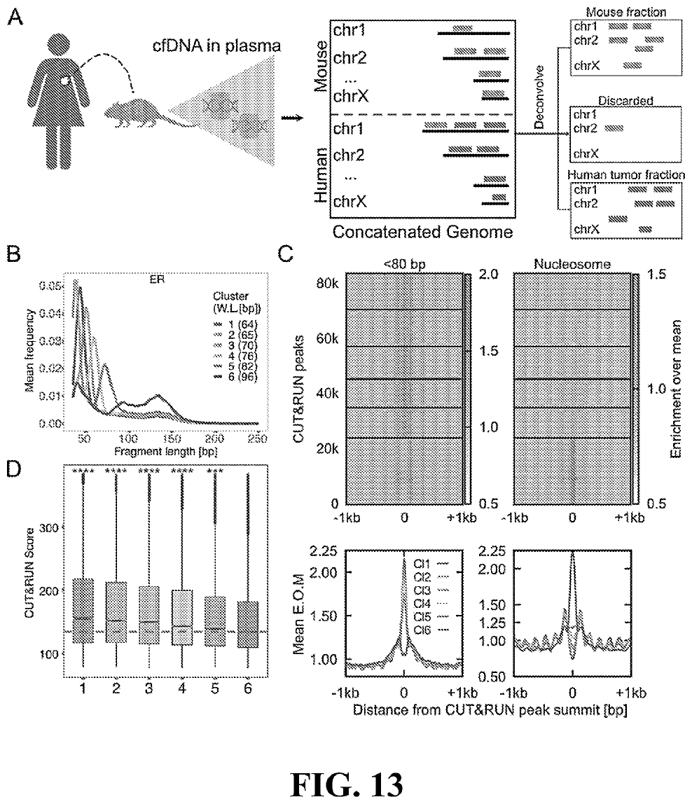

In cancer patients, cancer cells also contribute significantly to plasma cfDNA. Hence, we hypothesized that cancer cell derived cfDNA will lead to dilution of lymphoid/myeloid signal. Such dilution would lead to a proportional decrease in enrichment of short fragments at Clusters 1 and 2 of hematopoietic TFBS due to cfDNA contributions from non-hematopoietic cell types where PU.1 and LYL1 are absent. To test this hypothesis, we performed k-means clustering of PU.1 and LYL1 binding sites based on the cfDNA length distributions for cfDNA from donors with cancer. We found that the short fragment enrichment for the bound clusters (1 and 2) was the highest for healthy human plasma (FIG. 12, panels D, E, I, and J). Cancer samples had significantly weaker short fragment enrichment at sites from clusters 1 and 2 for PU.1 and LYL1 (FIG. 12, panels E, J) and did not have higher ChIP scores compared to cluster 6. In addition to using cfDNA from cancer patients, we also used human cfDNA from cell-line/patient-derived xenografts (C/PDXs) (FIG. 13, panel A). Since the only source of human cfDNA in a xenograft is from the cancer cells, fragments that uniquely map to the human genome in this context represent pure circulating tumor DNA (ctDNA). We found no expression of PU.1 or LYL1 in breast tumor model systems, and accordingly, we observed no nucleosome phasing or higher ChIP scores for the top 2 clusters in the xenograft cfDNA. Additionally, we found an expected decrease in enrichment of short fragments in clusters 1 and 2 from the xenografts when compared to healthy donor (FIG. 12, panels D, I, E, and J; sample names: UCD65 and MCF7). The clear separation between cfDNA from a healthy donor and cfDNA from cancer patients and from xenografts suggest that the length profiles of cfDNA at hematopoietic TFBS when combined with local enrichment of short fragments can identify dilution of lymphoid/myeloid cfDNA across diverse plasma samples.

### ctDNA Maps Tumor-Specific TF Binding

We were able to uncover strong signals of CTCF and hematopoietic TFs binding in plasma cfDNA because the vast majority of cells that release cfDNA have these TFs bound in their genome. However, tumor-specific TFs will, by definition, have weaker signals because tumor cfDNA is always a minor fraction of total cfDNA. In order to develop pure tumor signatures of TF binding in cfDNA, we turned to human cancer xenografts implanted in mice. Since the tumor-derived cfDNA in PDX would map to the human genome, and the endogenous cfDNA from the mouse would map to the mouse genome, we could identify cfDNA molecules from sequencing that were purely from the tumor, hence circulating tumor DNA (ctDNA), but obtained from a closed in vivo system (FIG. 13, panel A). We used ER+ breast tumor cells, UCD65 (50) and MCF7, as ER+ tumors are driven by the TFs Estrogen Receptor (ER) and FOXA1. We first profiled ER and FOXA1 binding using CUT&RUN (19). CUT&RUN is an alternative to ChIP-seq that relies on a protein-A-tagged nuclease that binds to a primary antibody of epitope of choice. The nuclease is activated upon addition of calcium, which results in the release of DNA fragments bound to ER. Due to the absence of crosslinking and release of bound sites rather than enrichment of bound sites, CUT&RUN captures TF binding at higher sensitivity and provides a greater dynamic range of signals compared to ChIP-seq (19). We performed CUT&RUN for ER and FOXA1 in estradiol (E2)-treated MCF7 cells and obtained ˜80,000 and ˜40,000 CUT&RUN sites for ER, and FOXA1 respectively, with sufficient coverage in our PDX cfDNA datasets (MCF7, UCD65).

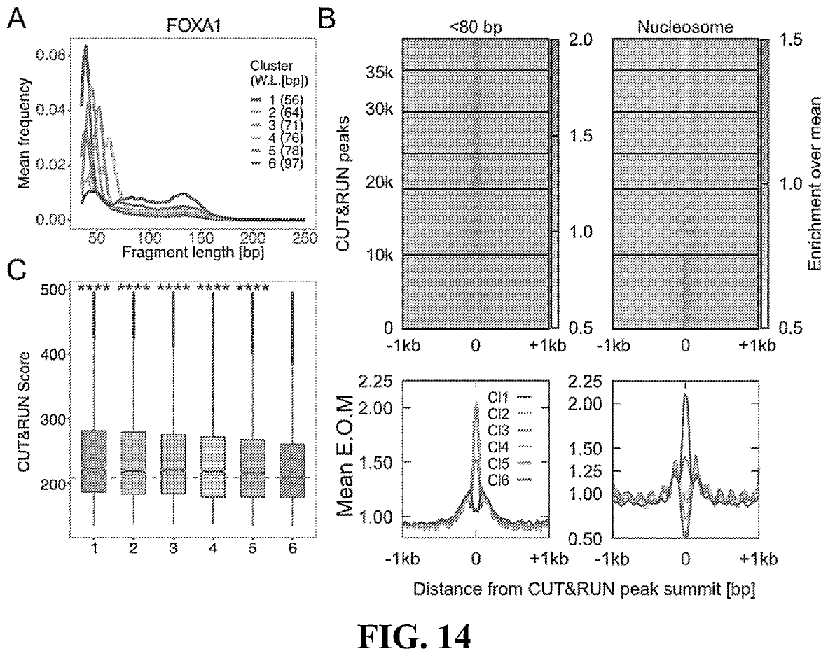

Importantly, when we performed fragment-length distribution analysis at ER CUT&RUN peaks and defined six clusters, the four clusters with lowest expected fragment length (FIG. 13, panel B) showed strong short fragment protections and phased nucleosomes (FIG. 13, panel C) as well as significantly higher ER binding measured as CUT&RUN score (FIG. 13, panel D). We observed similar trends for FOXA1 binding sites (FIG. 14, panel A-C). Positive correlation between ctDNA short fragment enrichment and CUT&RUN scores strongly suggests that we are capturing binding in cancer cells and that the signal from cfDNA release in vivo is similar to CUT&RUN profiling. Thus, defining binding sites in tumor cells using CUT&RUN enables sensitive mapping in plasma of the TF-binding that occurs in tumor-cells-of-origin.

### Unique Sets of TFBS Display Tissue-of-Origin-Specific TF Protections in Plasma

We have defined sets of binding sites that show TF-specific protections in two pure systems: healthy plasma and PDX plasma. We now asked if we could define subset of these sites that would be unique to the tissue-of-origin. To do this, we performed length clustering analysis at all TFBS with both healthy plasma dataset and with the PDX datasets to identify binding site clusters with significantly higher ChIP/CUT&RUN binding scores compared to the nucleosomal cluster of binding sites for each cfDNA dataset. We then intersected the significant binding sites between healthy plasma and PDX models. First, we found that PU.1 and LYL1 sites had TF protections that correlated with binding strength only in healthy plasma (FIG. 15, panel A), indicating that all significant TFBS of PU.1 and LYL1 could be used to identify hematopoietic contribution to cfDNA. CTCF is a constitutive factor, ER is expressed in T cells (51, 52), and factors related to FOXA1 that have same binding motifs are expressed in hematopoietic cells, for example, FOXM1 (53-55). The partial overlap of binding of these or related factors in hematopoietic and cancer cells led to us finding sites with significant TF protections in both healthy plasma and in PDX for CTCF, FOXA1, and ER (FIG. 15, panel A, and data not shown). For example, a large fraction of sites of CTCF (16709 in set 2 and 4945 in set 4) are shared between PDX and healthy plasma. Rest of the CTCF sites (17902 in set 1, 6022 in set 3, 4930 in set 5, and 4649 in set 6, CTCF in FIG. 15, panel A) are cancer specific. In contrast, the top 3 sets of sites for FOXA1 and ER are PDX-specific, with the largest set of sites specific to UCD65 (8226 for FOXA1 and 13879 for ER). FOXA1 has sites specific to MCF7 as well (set 3) and ER has sites specific to MCF7 (set 3) and UCD4 (set 6). Thus, in spite of overlap in binding between hematopoietic cells and cancer cells, ER and FOXA1 have enough unique sites protected in plasma that not only distinguish healthy plasma from PDX, but also distinguish individual PDXs.

Although FOXA1 is not expressed in lymphoid/myeloid cells, some FOXA1 binding sites identified in MCF7 cells showed significant enrichment of TF footprints in healthy plasma. We asked if related FOX factors like FOXM1 and FOXK2 that are expressed in lymphoid/myeloid cells may be binding at these sites to give rise to short footprints in cfDNA. To ask if FOXM1 or FOXK2 give rise to footprints at a subset of FOXA1 sites, we calculated scores for FOXM1 and FOXK2 binding from ChIP experiments conducted in GM12878 cells. We found FOXM1 ChIP scores to strongly correlate with short length clusters in healthy plasma but not FOXK2 ChIP scores. This indicates that FOXM1 occupies sites in lymphoid/myeloid cells that are a subset of sites bound by FOXA1 in MCF7 cells.

With these collections of sites that were unique to cancer and to the ER status (normal vs. amplified vs. mutated), we calculated a plasma TF binding score: the number of short reads (<80 bp) mapped within 50 bp of the TFBS normalized by the number of reads in 1000 bp around the TFBS. This plasma TF score tracks with the identity of the sites: the sites unique to healthy plasma had a significantly higher TF score for healthy plasma compared to PDX and vice versa. Similarly, sites specific to UCD65, MCF7, and UCD4 when compared to each other also had higher plasma TF scores (FIG. 15, panels B, D, E, and F). Thus, unique sets of sites identified using cfDNA length clusters also had localized enrichment of short fragments relative to the surrounding 1000 bp in a system-specific manner, which shows the potential of cfDNA length clusters to identify not only the tissue-of-origin but also the disease state.

In a plasma sample from an individual with cancer, both lymphoid/myeloid cells and tumor cells will contribute to cfDNA, with majority of the contribution still being from the lymphoid/myeloid cells. To ask at what dilution of tumor DNA we could detect the presence of cancer using TF footprints, we performed in silico dilutions of PDX cfDNA, which represents pure tumor DNA into healthy plasma cfDNA at 0, 0.5, 1, 2, 3, 4, and 5%. We then calculated plasma TF binding score at sites specific to healthy plasma and PDX. We compared these scores between the in silico diluted plasma samples and non-diluted plasma sample to calculate a paired t-statistic. We set a cut-off of 5 for the median paired t-statistic to indicate a significant difference between diluted and non-diluted plasma sample. We found ER sites to be strongest in separating tumor diluted cfDNA from pure healthy cfDNA (detection at <1% tumor cfDNA) followed by FOXA1 and CTCF (detection at ˜1% of tumor cfDNA, FIG. 15, panel C). PU.1 (detection at 2% tumor cfDNA) and LYL1 had weaker but significant contributions (not shown). Combined ER and FOXA1 sites showed a median t-statistic greater than 5 between 0.5 and 1% tumor fraction. Since most metastatic disease states have tumor fractions higher than 1% (56, 57), our analysis suggests that we would be able to delineate TF binding in metastatic tumors, in spite of the significant interference from cfDNA of lymphoid/myeloid origin.

We next asked if we could differentiate between the PDXs based on their ER status: ER expression is much higher in UCD65 (ESR1 amplification) and UCD4 has a mutated ER (activating D538G mutation) (58). Both ER and FOXA1 sites contribute to differentiating UCD65 from MCF7. Combining sites from both TFs is synergistic and separates UCD65 and MCF7 at 4% of tumor fraction (t-statistic>5, FIG. 15, panel G). Thus, at marginally higher tumor fractions, we can even identify signatures of differences in ER expression levels using TFBS defined by a combination of CUT&RUN and cfDNA length clustering. Strikingly, ER sites could robustly differentiate UCD4 from UCD65 and MCF7 (FIG. 15, panels H, I), highlighting the fact that mutated ER leads to differential binding signature that can be identified in plasma cfDNA at 2% tumor fraction. Significantly, FOXA1 sites were much weaker than ER in differentiating UCD4 from UCD65 and MCF7, highlighting that the mutation-specific changes in TF footprints in plasma is strongest for ER. In summary, by identifying the subset of high-resolution TFBS protected in distinct plasma samples, we are able to define TF signatures unique to ER+ breast cancer and further, unique to amplified WT ER and ER D538G.

### Identified TFBS Report on Tumor TF Binding in Individuals With Breast Cancer

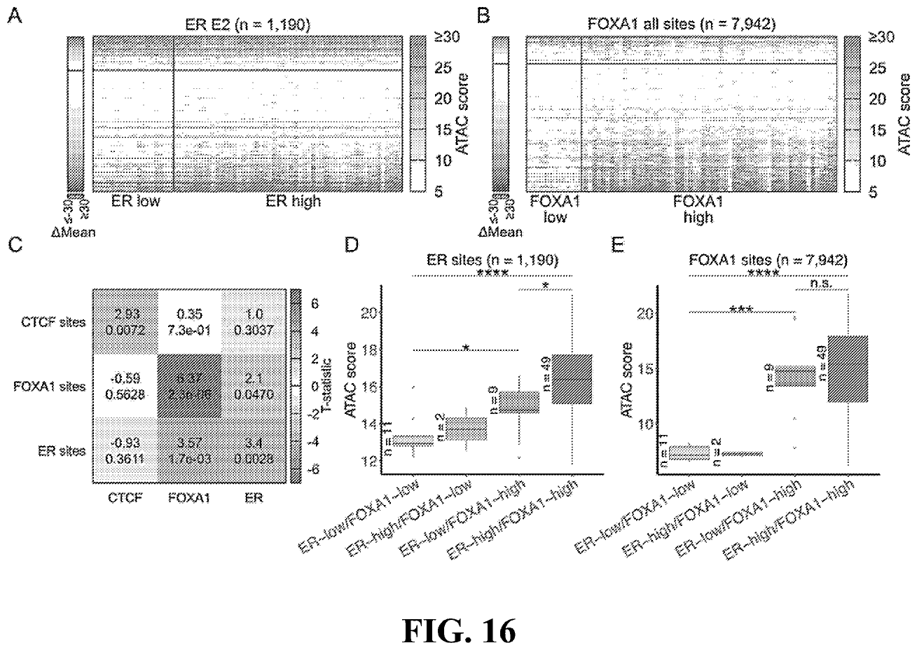

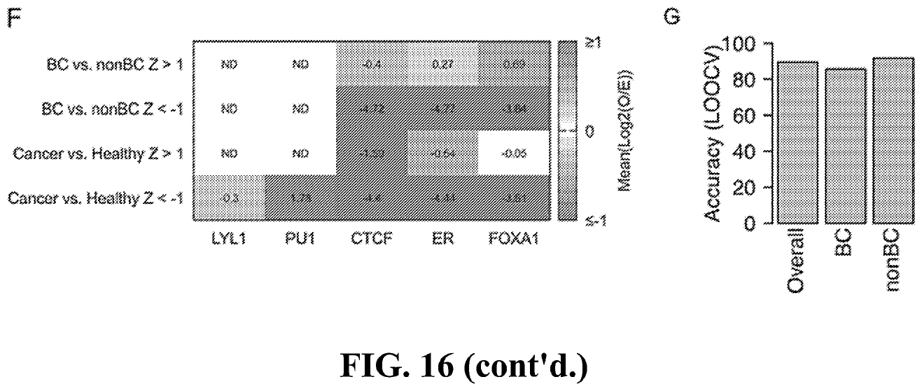

Since our in silico dilution analyses indicate that TF footprints in plasma can identify breast cancer disease state at tumor fractions of 1-4%, we next asked if the TFBSs we identified to be uniquely protected in PDX plasma would reflect disease states in heterogeneous human samples. To test this, we first turned to ATAC-seq datasets generated using primary tumor samples in the TCGA database. ATAC-seq reports on DNA accessibility, which highly correlates with TF binding (59). We asked if tumors exhibited TF-specific accessibility at the TFBSs we identified. We ordered BRCA tumors based on a specific TF expression and then calculated accessibility at sites identified to be UCD65-specific. We found tumors that express ER (Transcripts Per Million (TPM)≥10) had a vast majority of UCD65-specific ER sites with higher accessibility compared too tumors that do not express ER (TPM<10, FIG. 16, panel A). We found even stronger accessibility differences at UCD65-specific FOXA1 binding sites, with FOXA1-expressing tumors having much higher ATAC scores than FOXA1-non-expressing tumors at a vast majority of sites (FIG. 16, panel B).

FOXA1 is known to act as a pioneer factor, enabling ER binding by establishing accessibility at its binding sites (34, 60). We asked if we could reproduce this finding at ER and FOXA1 binding sites we identified by taking advantage of the heterogeneity in ER and FOXA1 expression across TCGA samples. If the ER and FOXA1 sites we identified are representative of ER and FOXA1 function across human breast tumors, then accessibility at ER binding sites should depend on the presence of FOXA1. CTCF is a good control as its expression should not influence accessibility at ER or FOXA1 sites. We first calculated the mean ATAC-score for each tumor sample by aggregating the ATAC score across all sites of a given TF. For CTCF, ER, and FOXA1 sites, we performed two sample t-test (sample 1: cohorts with high TF expression (top 15), sample 2: cohorts with low TF expression (bottom 15)). We found the mean ATAC-scores at CTCF, FOXA1, and ER sites were significantly different when tumors were grouped by the expression of the respective TF, with strongest difference seen for FOXA1 (diagonal cells in FIG. 16, panel C). Strikingly, we observed a strong difference (t-statistic=3.57; p=1.7×10⁻³) in mean ATAC-scores at ER sites when tumors were grouped based on FOXA1 expression. This difference was stronger than at FOXA1 sites when tumors were grouped based on ER expression (t-statistic=2.1; p=0.047), suggesting that FOXA1 expression has a stronger influence on accessibility at ER sites than vice versa.

To further explore the effect of FOXA1 at ER sites, we stratified BRCA tumors by both ER and FOXA1 expression levels. In tumors with low ER expression, increase in FOXA1 expression led to a significant increase in mean ATAC-scores at ER sites, suggesting that FOXA1 keeps the chromatin open at ER sites even in the absence of ER (FIG. 16, panel D). Expression of ER and FOXA1 led to the highest accessibility at ER sites suggesting further chromatin opening post ER binding (FIG. 16, panel D). In stark contrast, at FOXA1 sites, accessibility increase is seen only due to increase in FOXA1 expression. The presence of ER did not lead to a significant increase in accessibility (FIG. 16, panel E). Our observation of FOXA1 expression driving accessibility at both ER and FOXA1 binding sites agrees well with the fact that FOXA1 is a pioneer factor that opens up ER sites. Taken together, our analysis shows that sites with tumor-specific plasma protections in PDXs can define TF-specific accessibility across human breast tumors. These results indicate that TF protections in plasma can define tumor TF binding in humans.

Next, we asked if TF binding scores from plasma cfDNA can distinguish cancer from healthy states and breast cancer from other cancers and healthy states. We compared TF binding scores in 19 human plasma cfDNA sequencing datasets (healthy=4, non-breast cancer=8 (total nonBC=12); breast cancer (BC)=7). To take advantage of samples that were sequenced at varying depths, we defined TF features as aggregates of 250 binding sites of the TF after ordering all its binding sites by ChIP/CUT&RUN score. We ended up with a total of 359 features (PU.1=43, LYL1=7, CTCF=120, ER=124, FOXA1=65). We made two classification groups: cancer vs. healthy (n=15,4) and BC vs. nonBC (n=7,12). We calculated the Z-score for each feature for these two groups of classification. We then filtered for those features with |Z|>1 in each of the two classifications as features that differentiated the two classes in each classification. We then asked which of the TFs had their features over-represented or under-represented in each classification. We found PU.1 features to be over-represented in having higher TF binding scores in healthy samples compared to cancer samples (FIG. 16, panel F). In classifying BC and nonBC, we found no TFs to be overrepresented in features that had higher binding scores in nonBC. However, ER and FOXA1 features were overrepresented with higher binding scores in BC compared to nonBC (FIG. 16, panel F). The fact that FOXA1 and ER binding sites can separate BC from nonBC indicates that the sites identified from PDXs are transferrable to human samples. Furthermore, in spite of dilution by cfDNA from lymphoid and myeloid cells, cancer-specific TF protections in plasma are sensitive markers of disease presence. To ask how accurate these features are in identifying presence of breast cancer, we resorted to leave-one-out cross validation. We identified features that significantly separated BC from nonBC using all but one of the samples (18 out of 19) and then used these features to predict status of the left-out sample. We observed an overall prediction accuracy of 89.5%, prediction accuracy of 85.7% for BC (6/7 predicted correctly), and accuracy of 91.7% for nonBC (11/12 predicted correctly, FIG. 16, panel G). Thus, our analysis with low to intermediate depth sequencing of 19 human plasma samples shows potential for plasma TF footprints to identify breast cancer tissue-of-origin.

### REFERENCES FOR EXAMPLE 2

1. K. Takahashi et al., Induction of pluripotent stem cells from adult human fibroblasts by defined factors. Cell 131, 861-872 (2007).
2. F. Spitz, E. E. Furlong, Transcription factors: from enhancer binding to developmental control. Nat Rev Genet 13, 613-626 (2012).
3. G. Damante et al., Sequence-specific DNA recognition by the thyroid transcription factor-1 homeodomain. Nucleic Acids Res 22, 3075-3083 (1994).
4. A. L. Todeschini, A. Georges, R. A. Veitia, Transcription factors: specific DNA binding and specific gene regulation. Trends Genet 30, 211-219 (2014).
5. Z. Wunderlich, L. A. Mirny, Different gene regulation strategies revealed by analysis of binding motifs. Trends Genet 25, 434-440 (2009).
6. T. W. Whitfield et al., Functional analysis of transcription factor binding sites in human promoters. Genome Biol 13, R50 (2012).
7. A. Mathelier, W. W. Wasserman, The next generation of transcription factor binding site prediction. PLoS Comput Biol 9, e1003214 (2013).
8. G. A. Jindal, E. K. Farley, Enhancer grammar in development, evolution, and disease: dependencies and interplay. Dev Cell 56, 575-587 (2021).
9. G. E. Ryan, E. K. Farley, Functional genomic approaches to elucidate the role of enhancers during development. Wiley Interdiscip Rev Syst Biol Med 12, e1467 (2020).
10. K. L. MacQuarrie, A. P. Fong, R. H. Morse, S. J. Tapscott, Genome-wide transcription factor binding: beyond direct target regulation. Trends Genet 27, 141-148 (2011).
11. S. A. Lambert et al., The Human Transcription Factors. Cell 172, 650-665 (2018).
12. A. Arvey, P. Agius, W. S. Noble, C. Leslie, Sequence and chromatin determinants of cell-type-specific transcription factor binding. Genome Res 22, 1723-1734 (2012).
13. S. L. Klemm, Z. Shipony, W. J. Greenleaf, Chromatin accessibility and the regulatory epigenome. Nat Rev Genet 20, 207-220 (2019).
14. T. C. Voss, G. L. Hager, Dynamic regulation of transcriptional states by chromatin and transcription factors. Nat Rev Genet 15, 69-81 (2014).
15. S. Ramachandran, S. Henikoff, Transcriptional Regulators Compete with Nucleosomes Post-replication. Cell 165, 580-592 (2016).
16. T. I. Lee, R. A. Young, Transcriptional regulation and its misregulation in disease. Cell 152, 1237-1251 (2013).
17. Y. Honaker et al., Gene editing to induce FOXP3 expression in human CD4(+) T cells leads to a stable regulatory phenotype and function. Sci Transl Med 12, (2020).
18. E. P. Consortium, An integrated encyclopedia of DNA elements in the human genome. Nature 489, 57-74 (2012).
19. P. J. Skene, S. Henikoff, An efficient targeted nuclease strategy for high-resolution mapping of DNA binding sites. Elife 6, (2017).
20. A. Barski et al., High-resolution profiling of histone methylations in the human genome. Cell 129, 823-837 (2007).
21. M. J. Rossi et al., A high-resolution protein architecture of the budding yeast genome. Nature 592, 309-314 (2021).
22. Y. M. Lo et al., Maternal plasma DNA sequencing reveals the genome-wide genetic and mutational profile of the fetus. Sci Transl Med 2, 61ra91 (2010).
23. M. W. Snyder, M. Kircher, A. J. Hill, R. M. Daza, J. Shendure, Cell-free DNA Comprises an In Vivo Nucleosome Footprint that Informs Its Tissues-Of-Origin. Cell 164, 57-68 (2016).
24. A. Zviran et al., Genome-wide cell-free DNA mutational integration enables ultra-sensitive cancer monitoring. Nat Med 26, 1114-1124 (2020).
25. M. C. Liu et al., Sensitive and specific multi-cancer detection and localization using methylation signatures in cell-free DNA. Ann Oncol 31, 745-759 (2020).
26. A. J. Bronkhorst, V. Ungerer, S. Holdenrieder, The emerging role of cell-free DNA as a molecular marker for cancer management. Biomol Detect Quantif 17, 100087 (2019).
27. P. Ulz et al., Inferring expressed genes by whole-genome sequencing of plasma DNA. Nat Genet 48, 1273-1278 (2016).
28. S. Ramachandran, K. Ahmad, S. Henikoff, Transcription and Remodeling Produce Asymmetrically Unwrapped Nucleosomal Intermediates. Mol Cell 68, 1038-1053 e1034 (2017).
29. P. Ulz et al., Inference of transcription factor binding from cell-free DNA enables tumor subtype prediction and early detection. Nat Commun 10, 4666 (2019).
30. Y. Y. Lui et al., Predominant hematopoietic origin of cell-free DNA in plasma and serum after sex-mismatched bone marrow transplantation. Clinical chemistry 48, 421-427 (2002).
31. H. Schwarzenbach, D. S. Hoon, K. Pantel, Cell-free nucleic acids as biomarkers in cancer patients. Nat Rev Cancer 11, 426-437 (2011).
32. F. Diehl et al., Detection and quantification of mutations in the plasma of patients with colorectal tumors. Proc Natl Acad Sci USA 102, 16368-16373 (2005).
33. J. Finlay-Schultz et al., Breast Cancer Suppression by Progesterone Receptors Is Mediated by Their Modulation of Estrogen Receptors and RNA Polymerase III. Cancer Res 77, 4934-4946 (2017).
34. A. Hurtado, K. A. Holmes, C. S. Ross-Innes, D. Schmidt, J. S. Carroll, FOXA1 is a key determinant of estrogen receptor function and endocrine response. Nat Genet 43, 27-33 (2011).
35. J. S. Carroll et al., Genome-wide analysis of estrogen receptor binding sites. Nat Genet 38, 1289-1297 (2006).
36. G. N. Filippova et al., An exceptionally conserved transcriptional repressor, CTCF, employs different combinations of zinc fingers to bind diverged promoter sequences of avian and mammalian c-myc oncogenes. Mol Cell Biol 16, 2802-2813 (1996).
37. S. J. Holwerda, W. de Laat, CTCF: the protein, the binding partners, the binding sites and their chromatin loops. Philos Trans R Soc Lond B Biol Sci 368, 20120369 (2013).
38. A. S. Hansen, I. Pustova, C. Cattoglio, R. Tjian, X. Darzacq, CTCF and cohesin regulate chromatin loop stability with distinct dynamics. Elife 6, (2017).
39. Y. Fu, M. Sinha, C. L. Peterson, Z. Weng, The insulator binding protein CTCF positions 20 nucleosomes around its binding sites across the human genome. PLoS Genet 4, e1000138 (2008).
40. C. T. Clarkson et al., CTCF-dependent chromatin boundaries formed by asymmetric nucleosome arrays with decreased linker length. Nucleic Acids Res 47, 11181-11196 (2019).
41. J. G. Henikoff, J. A. Belsky, K. Krassovsky, D. M. MacAlpine, S. Henikoff, Epigenome characterization at single base-pair resolution. Proc Natl Acad Sci USA 108, 18318-18323 (2011).
42. P. Burda, P. Laslo, T. Stopka, The role of PU.1 and GATA-1 transcription factors during normal and leukemogenic hematopoiesis. Leukemia 24, 1249-1257 (2010).
43. R. C. Fisher, E. W. Scott, Role of PU.1 in hematopoiesis. Stem Cells 16, 25-37 (1998).
44. S. K. Chiu et al., A novel role for Lyl1 in primitive erythropoiesis. Development 145, (2018).
45. K. L. Davis, Ikaros: master of hematopoiesis, agent of leukemia. Ther Adv Hematol 2, 359-368 (2011).
46. J. Zhu, S. G. Emerson, Hematopoietic cytokines, transcription factors and lineage commitment. Oncogene 21, 3295-3313 (2002).
47. I. Barozzi et al., Coregulation of transcription factor binding and nucleosome occupancy through DNA features of mammalian enhancers. Mol Cell 54, 844-857 (2014).
48. M. Iwafuchi-Doi, K. S. Zaret, Pioneer transcription factors in cell reprogramming. Genes Dev 28, 2679-2692 (2014).
49. J. N. Wu et al., Functionally distinct patterns of nucleosome remodeling at enhancers in glucocorticoid-treated acute lymphoblastic leukemia. Epigenetics Chromatin 8, 53 (2015).
50. P. Kabos et al., Patient-derived luminal breast cancer xenografts retain hormone receptor heterogeneity and help define unique estrogen-dependent gene signatures. Breast Cancer Res Treat 135, 415-432 (2012).
51. I. Mohammad et al., Estrogen receptor alpha contributes to T cell-mediated autoimmune inflammation by promoting T cell activation and proliferation. Sci Signal 11, (2018).
52. D. H. Kim et al., Estrogen receptor alpha in T cells suppresses follicular helper T cell responses and prevents autoimmunity. Exp Mol Med 51, 1-9 (2019).
53. S. Uddin et al., Overexpression of FoxM1 offers a promising therapeutic target in diffuse large B-cell lymphoma. Haematologica 97, 1092-1100 (2012).
54. Y. Sheng et al., FOXM1 regulates leukemia stem cell quiescence and survival in MLL-rearranged AML. Nat Commun 11, 928 (2020).
55. C. Gu et al., FOXM1 is a therapeutic target for high-risk multiple myeloma. Leukemia 30, 873-882 (2016).
56. R. J. Leary et al., Detection of chromosomal alterations in the circulation of cancer patients with whole-genome sequencing. Sci Transl Med 4, 162ra154 (2012).
57. V. A. Adalsteinsson et al., Scalable whole-exome sequencing of cell-free DNA reveals high concordance with metastatic tumors. Nat Commun 8, 1324 (2017).
58. J. Finlay-Schultz et al., New generation breast cancer cell lines developed from patient-derived xenografts. Breast Cancer Res 22, 68 (2020).
59. M. R. Corces et al., The chromatin accessibility landscape of primary human cancers. Science 362, (2018).
60. S. E. Glont, I. Chernukhin, J. S. Carroll, Comprehensive Genomic Analysis Reveals that the Pioneering Function of FOXA1 Is Independent of Hormonal Signaling. Cell Rep 26, 2558-2565 e2553 (2019).
61. A. Zukowski, S. Rao, S. Ramachandran, Phenotypes from cell-free DNA. Open Biol 10, 200119 (2020).
62. M. Uhlen et al., A pathology atlas of the human cancer transcriptome. Science 357, (2017).
63. C. S. Ross-Innes, G. D. Brown, J. S. Carroll, A co-ordinated interaction between CTCF and ER in breast cancer cells. BMC Genomics 12, 593 (2011).
64. J. Cheneby et al., ReMap 2020: a database of regulatory regions from an integrative analysis of Human and Arabidopsis DNA-binding sequencing experiments. Nucleic Acids Res 48, D180-D188 (2020).
65. O. Fornes et al., JASPAR 2020: update of the open-access database of transcription factor binding profiles. Nucleic Acids Res 48, D87-D92 (2020).
66. I. V. Kulakovskiy et al., HOCOMOCO: towards a complete collection of transcription factor binding models for human and mouse via large-scale ChIP-Seq analysis. Nucleic Acids Res 46, D252-D259 (2018).
67. B. Langmead, S. L. Salzberg, Fast gapped-read alignment with Bowtie 2. Nat Methods 9, 357-359 (2012).
68. A. Savitzky, M. J. E. Golay, Smoothing and Differentiation of Data by Simplified Least Squares Procedures. Analytical Chemistry 36, 1627-1639 (1964).
69. C. R. Harris et al., Array programming with NumPy. Nature 585, 357-362 (2020).
70. H. Li et al., The Sequence Alignment/Map format and SAMtools. Bioinformatics 25, 2078-2079 (2009).
71. C. E. Grant, T. L. Bailey, W. S. Noble, FIMO: scanning for occurrences of a given motif. Bioinformatics 27, 1017-1018 (2011).
72. A. R. Quinlan, I. M. Hall, BEDTools: a flexible suite of utilities for comparing genomic features. Bioinformatics 26, 841-842 (2010).
73. W. J. Kent, A. S. Zweig, G. Barber, A. S. Hinrichs, D. Karolchik, BigWig and BigBed: enabling browsing of large distributed datasets. Bioinformatics 26, 2204-2207 (2010).
74. F. Ramirez, F. Dundar, S. Diehl, B. A. Gruning, T. Manke, deepTools: a flexible platform for exploring deep-sequencing data. Nucleic Acids Res 42, W187-191 (2014).
75. A. Lex, N. Gehlenborg, H. Strobelt, R. Vuillemot, H. Pfister, UpSet: Visualization of Intersecting Sets. IEEE Trans Vis Comput Graph 20, 1983-1992 (2014).

### EXAMPLE 3

### cfDNA Subnucleosome and Nucleosome Analysis for Uncovering Disease State and Immune Response in ER+ Breast Cancer and NSCLC

There is an interest in exploiting the chromatin structural information in cell free DNA (cfDNA) to map cancer phenotype. Cell free DNA (cfDNA) is a rich source of genetic and epigenetic information that can be obtained in a minimally invasive manner from patient blood samples. Current clinical cfDNA applications focus on identifying oncogenic mutations. However, mutations are only a small subset of the information that is contained in cfDNA. cfDNA is generated by action of endogenous nucleases on a chromatinized genome, which means that cfDNA is essentially a map of chromatin structure of their originating cells (1). A genome-wide map of chromatin structure can reveal the regulatory landscape of the cell and provides a richer tapestry of information compared to mutation panels. Furthermore, chromatin structure reflects cellular identity (2). Knowledge of how chromatin structure is connected to cell states will enable us to extract tissue-of-origin information from cfDNA, unlocking additional layers of information from the same source.

Epigenomic signatures from plasma cell-free DNA (cfDNA) have been proposed as biomarkers for tracking disease states. In a healthy person, cfDNA is generated by normal turnover of lymphoid and myeloid tissue. From the onset of tumorigenesis, tumor cells also contribute to cfDNA. It has been shown that cfDNA offers unprecedented insights into cancer physiology (3). As such, combined signatures of the immune system and the tumor in a patient, as defined by cfDNA epigenomics, can predict and track treatment response and disease states. The basis comes from an important observation that short cfDNA fragments in plasma (less than the minimum length needed to wrap around histone octamer, so called “subnucleosomal fragments”) represent transcription factor footprints (3) and nucleosome disassembly or re-assembly that accompany active transcription (4). In other words, these short “subnucleosome” DNA fragments enabled us to identify, define, and in turn predict the gene expression signatures of lymphoid/myeloid tissue in cfDNA from healthy donors, and importantly, detect dramatic changes in cfDNA signatures from cancer patients (3, 4). Thus, subnucleosome analysis at regulatory sites can not only help us understand the disease landscape that is amenable to treatment, but also lead to minimally invasive biomarkers.

### Predicting Treatment Response to NSCLC using cfDNA Subnucleosome Profiles

Immune checkpoint inhibitors (ICI) have revolutionized cancer therapy. They have been approved for multiple tumor types and can provide dramatic survival benefits and even long-term control of disease, in the treatment of melanoma, non-small cell lung cancer (NSCLC), and other solid tumors. An adaptive immune response countered by immune evasion by the tumor sets the stage for effective ICI (5). Tumors evade adaptive immune response by expressing PD-L1 which binds PD-1 in CD8⁺ T cells and inhibits their anti-tumor activity. Hence, the presence of PD-L1 on the tumor is used to select patients for treatment with PD-1/PD-L1 inhibitors. In some cases, 1% of PD-L1 immunohistochemistry (IHC) staining on tumor cells is considered sufficient for clinical use of immunotherapy (6). However, ˜55% of patients selected using PD-L1 staining do not benefit (7, 8), while potentially suffering from side effects. On the other hand, it is evident that therapy is being denied to patients who may benefit but do not show clear PD-L1 staining at the time of selection (9). The risks associated with ICI-related adverse events, mixed performance of PD-L1 staining in predicting treatment response, and its high cost presents a clinical need for more precise methods to define disease states in the context of ICI treatment.

Epigenomic signatures from plasma cell-free DNA (cfDNA) is an alternative in view of that which is described herein. Current liquid biopsy approaches measure cancer genotypes but are blind to changes in immune component of cfDNA. However, ICI response is thought to depend on the phenotype of the tumor and the associated immune response, especially functional state of CD8⁺ T cells. As such, the combined signatures of the immune system and the tumor in a patient, as defined by cfDNA epigenomics, can predict and track response to ICI. To understand and predict tumor and immune states that enable ICI to stop cancer progression, plasma cfDNA samples collected have been sequenced prior to start of treatment of NSCLC with the PD-1 inhibitor, pembrolizumab. These samples have been collected as part of an ongoing clinical trial, and participants' response to treatment is known. Below, interim analysis of this study is presented.

Sequencing of cfDNA was performed on 21 plasma samples from patients who had been treated with pembrolizumab as a first line treatment for metastatic NSCLC. Blood samples were drawn just before the first dose, 1 day to 1 week before the start of treatment. The treatment duration varied depending on response. Response was evaluated by CT scans every 8-12 weeks. 11 of the samples are from patients with no or minor response (<6 months of treatment), and 10 are from patients with prolonged benefit of the medication (>1 year of treatment). Since cfDNA is highly nicked, shorter fragments, which are most important for our analyses, are lost during standard library preparation. Hence, sequencing libraries were prepared from cfDNA that were denatured into single stranded DNA using the Single Strand Protocol (SSP) (1), which also captured all fragment lengths. Paired end sequencing was then performed to obtain an average of 100×106 reads per sample. These data were mapped back to the human genome, which provided both the location of the fragment in the genome and its length. Satisfactory fragment length distributions was obtained genome-wide from the cfDNA sequencing data, indicating that chromatin protections in cfDNA were being captured.

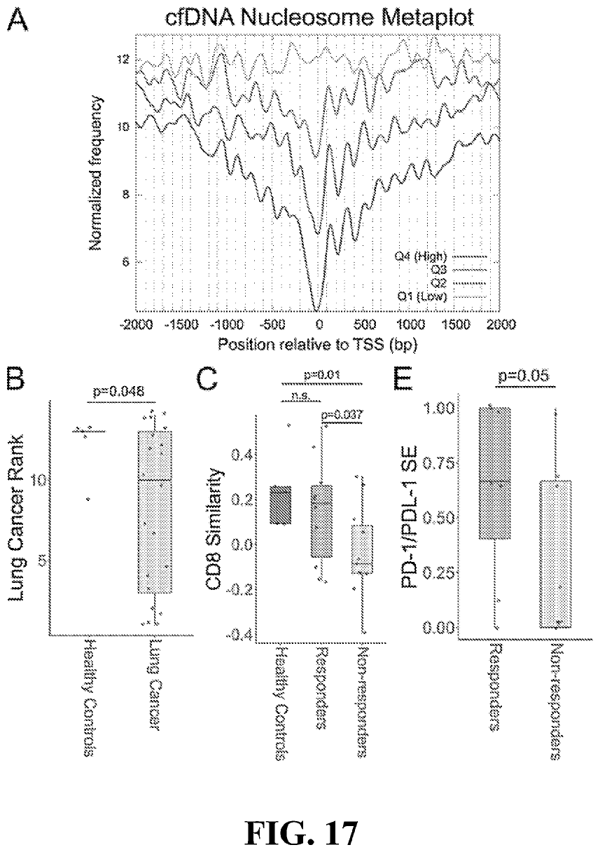

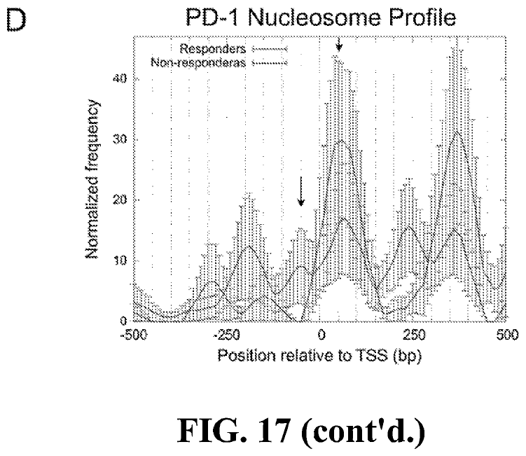

Since most of the cfDNA is contributed by hematopoietic cells, it was reasoned that cfDNA chromatin maps should reflect that of hematopoietic cells even in a cancer patient. Nucleosome-length fragments were computationally extracted from a representative NSCLC plasma sample and plotted their density around transcription start sites (TSS). Genes were stratified into quartiles based on expression levels of neutrophils as these cells have high rate of turnover in humans and are thought to significantly contribute to cfDNA. The average distribution of 155-170 bp fragments were plotted for each quartile (FIG. 17, panel A). A depletion of nucleosomes was observed at the TSS and ordered nucleosome arrays upstream and downstream of the TSS for the genes in the top quartile. An overall depletion of fragments in gene bodies at higher quartiles compared to lower quartiles was also observed. These are classical features of chromatin structure in expressed genes. In expressed genes, the transcription machinery assembles at the TSS, resulting in the depletion of nucleosomes. Similarly, expressed genes have much more accessible chromatin, hence are preferentially digested by nucleases, resulting in lower recovery of expressed regions compared to non-expressed regions. Since cfDNA fragments of nucleosomal length capture these key chromatin structural features in hematopoietic cell types, it was concluded that these cfDNA datasets represent chromatin structure of the cells that gave rise to the cfDNA. It was then asked if the presence of tumor could be detected using SE scores. Subnucleosome enrichment (SE) scores from cfDNA were correlated to expression profiles of 12 hematopoietic cell types using RNA-seq data derived from Hemopedia (10), and adenocarcinoma (AC) expression data from TCGA (11) averaged across 24 tumor samples to generate a representative expression profile. AC profiles were chosen since the vast majority of our samples are from patients with AC. For each sample (5 healthy controls, 21 NSCLC plasma samples), Ordered match between SE and AC expression profile were ranked. A higher rank indicates worse match to AC compared to hematopoietic cells. The healthy controls were observed to have high ranks and the NSCLC samples were observed to have significantly lower ranks, indicating that SE has better match to AC expression in the NSCLC datasets compared to healthy control datasets (FIG. 17, panel B). Thus, SE can detect presence of lung cancer in cfDNA datasets from cancer patients.

An active adaptive immune response to tumor is characterized by infiltration of CD8⁺ T cells. Accordingly, responders to ICI have higher levels of CD8⁺ T cells in the tumor microenvironment compared to non-responders (5). However, flow cytometry analysis of circulating leukocytes does not show elevated levels of PD-1⁺ CD8⁺ T cells in patients who respond to ICI (12). T cell turnover at tumor sites could release cfDNA. Thus, cfDNA could show CD8⁺ T cell signatures that are invisible to flow cytometry. To test this idea, the SE match was compared to expression profiles of CD8⁺ T cells in healthy controls, and NSCLC patients who either responded or did not respond to pembrolizumab treatment. No significant difference in CD8⁺ T cell similarity scores between healthy controls and responders was found (FIG. 17, panel C). However, the non-responders had significantly lower CD8⁺ T cell similarity scores compared to both responders and to healthy controls, in samples collected prior to treatment (FIG. 17, panel C). The lower match between cfDNA SE and CD8⁺ T cell expression in non-responders suggests that the immune response to tumor was weak prior to treatment, which could explain why ICI treatment did not stop disease progression. These results highlight the power of cfDNA SE to capture the immune response to disease in addition to cancer state itself.

Since pembrolizumab targets PD-1, it was next asked if nucleosome profiles could be used to infer PD-1 expression from cfDNA. When the nucleosome profiles for PD-1 gene were plotted, nucleosome depletion was observed at the promoter (upstream of TSS) and ordered nucleosomes downstream of the TSS for responders (FIG. 17, panel D). Strikingly, non-responders had significantly higher nucleosome occupancy at the promoter, and overall, more uniform density across the gene body. Comparing the cfDNA nucleosome profiles suggests higher PD-1 expression in immune cells of responders compared to non-responders in samples collected prior to start of ICI treatment. Higher PD-1 expression suggests that responders were primed for ICI treatment compared to non-responders, and this could be discerned directly from cfDNA. To ask if cfDNA SE can separate responders and non-responders based on PD-1/PD-L1 chromatin structure, a combined SE score was calculated for PD-1 and PD-L1 for responders and non-responders. PD-1/PD-L1 SE score was significantly higher in responders compared to non-responders (FIG. 17, panel E). PD-L1 IHC was performed for all patients prior to start of therapy and all but 2 patients in this cohort had PD-L1 staining>50%. However, the PD-L1 levels inferred by IHC was not significantly different between responders and non-responders (p=0.12). Taken together, our results suggest that promoter cfDNA subnucleosomes report on PD-1/PD-L1 status with higher sensitivity than IHC. In summary, cfDNA chromatin structure at promoters predicts response of NSCLC to pembrolizumab treatment.

Immune transcription factor footprints from cfDNA distinguish responders and non-responders prior to treatment. Apart from promoter dynamics, it has been shown that cfDNA can directly capture TF footprints (3). It was asked if the regulatory landscape of immune cells, including tumor infiltrating lymphocytes (TILs), could be captured from our NSCLC datasets. To identify reference TF binding sites in CD8⁺ T cells in an unbiased manner, we turned to ATAC-seq analysis performed by a collaborator (13). Clustering of publicly available ATAC-seq peaks from naïve, PD-1^{hi} TILs, memory T cells and exhausted T cells identified sites that were unique to naïve and PD-1^{hi} TILs. These clusters had enrichment for specific transcription factor (TF) motifs—binding sites unique to naïve T-cells were enriched for ETS and TCF7 motifs, whereas PD-1^{hi} TILs were enriched for AP-1, IRF family, and NFAT motifs.

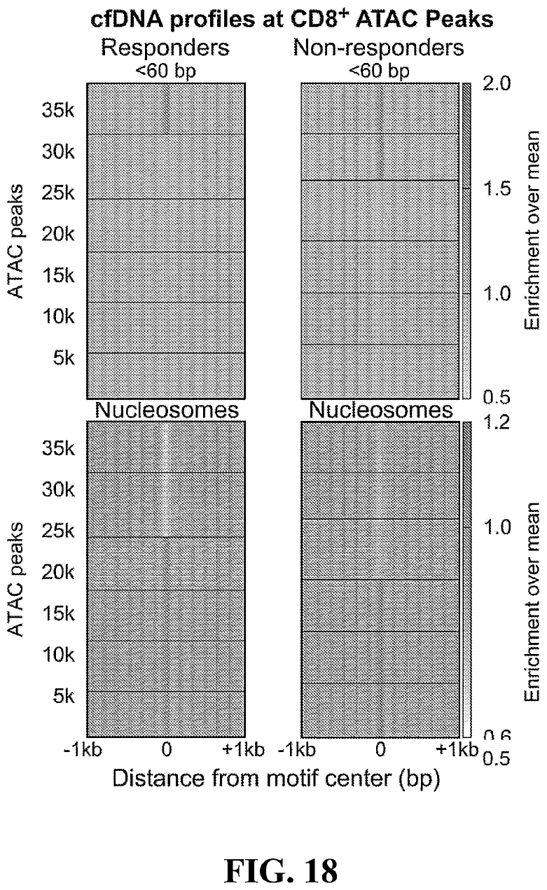

It was next asked if cfDNA TF footprints can be identified at these CD8⁺ T cell binding sites. At each ATAC peak, up to 5 motifs were mapped. At each motif, combined cfDNA fragment midpoints were mapped from all responders and all non-responders to estimate a fragment length distribution. K-means clustering of these fragment length distributions identified two types of clusters—one enriched with short cfDNA fragments (<100 bp) and the other enriched with long cfDNA fragments (>120 bp). When enrichment of cfDNA fragments around 1 kb of the motifs was mapped, cluster 1 had strong enrichment of short protections at motifs relative to 1 kb upstream and downstream of the motifs for both responders and non-responders (FIG. 18, top). Strikingly, these clusters also showed strong nucleosome phasing at least 1 kb upstream and downstream of the motifs (FIG. 18, bottom). Thus, fragment length profile at immune TF binding sites not only identified TF binding, but also uncovered chromatin structure surrounding the bound TF from plasma cfDNA.

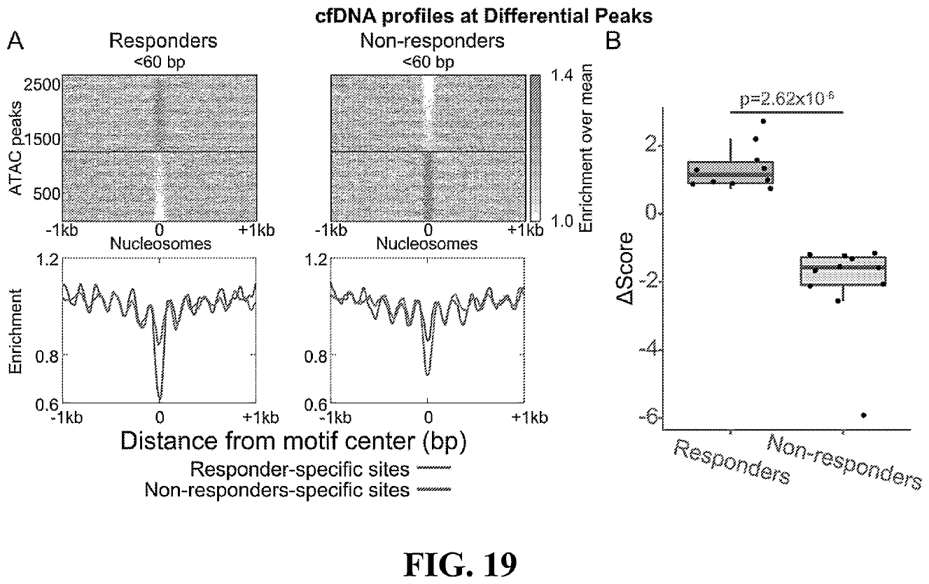

We then compared the enrichment of TF footprints for responders and non-responders to identify 1401 binding sites that had significantly stronger footprints in responders and 1274 binding sites that had significantly stronger footprints in non-responders (FIG. 19, panel A, top). Significantly, all these sites had phased nucleosomes for both responders and non-responders, but responder-specific sites had higher nucleosome depletion in responder cfDNA data and vice versa (FIG. 19, panel A, bottom). Though sites were selected only based on TF footprints, the corresponding change in nucleosome depletion further confirms that these sites represent TF binding.

To ask how well these sites separate the responders and non-responders, a composite delta score was calculated for each patient: enrichment of TF footprints at non-responder-specific sites was aggregated and subtracted this from the aggregated enrichment of TF footprints at responder-specific sites for each individual patient. A positive delta score will identify responders and a negative delta score will identify non-responders. This is exactly what was found—there is a striking separation between responders and non-responders that is highly statistically significant (FIG. 19, panel B). The top motifs that separate responders and non-responders are ETS1, IRF3, NFAC1, and TCF7, which are all enriched at ATAC peaks unique to naïve CD8⁺ T cells and PD-L1^{hi} TILs. Thus, cfDNA TF footprints are able to track the regulatory landscape of immune cells engaging with tumor. Further, TF footprint enrichment can be used to predict response to PD-1 inhibition. In summary, our pilot studies on NSCLC plasma samples collected prior to treatment from 21 patients demonstrate the power of cfDNA subnucleosome and nucleosome analysis to uncover both disease state and immune response in a single, minimally invasive assay.

### REFERENCES FOR EXAMPLE 3

1. Snyder M W, Kircher M, Hill A J, Daza R M, Shendure J. Cell-free DNA Comprises an In Vivo Nucleosome Footprint that Informs Its Tissues-Of-Origin. Cell. 2016; 164 (1-2):57-68. doi: 10.1016/j.cell.2015.11.050. PubMed PMID: 26771485; PMCID: PMC4715266.
2. Roadmap Epigenomics C, Kundaje A, Meuleman W, Ernst J, Bilenky M, Yen A, Heravi-Moussavi A, Kheradpour P, Zhang Z, Wang J, Ziller M J, Amin V, Whitaker J W, Schultz M D, Ward L D, Sarkar A, Quon G, Sandstrom R S, Eaton M L, Wu Y C, Pfenning A R, Wang X, Claussnitzer M, Liu Y, Coarfa C, Harris R A, Shoresh N, Epstein C B, Gjoneska E, Leung D, Xie W, Hawkins R D, Lister R, Hong C, Gascard P, Mungall A J, Moore R, Chuah E, Tam A, Canfield T K, Hansen R S, Kaul R, Sabo P J, Bansal M S, Carles A, Dixon J R, Farh K H, Feizi S, Karlic R, Kim A R, Kulkarni A, Li D, Lowdon R, Elliott G, Mercer T R, Neph S J, Onuchic V, Polak P, Rajagopal N, Ray P, Sallari R C, Siebenthall K T, Sinnott-Armstrong N A, Stevens M, Thurman R E, Wu J, Zhang B, Zhou X, Beaudet A E, Boyer L A, De Jager P L, Farnham P J, Fisher S J, Haussler D, Jones S J, Li W, Marra M A, McManus M T, Sunyaev S, Thomson J A, Tlsty T D, Tsai L H, Wang W, Waterland R A, Zhang M Q, Chadwick L H, Bernstein B E, Costello J F, Ecker J R, Hirst M, Meissner A, Milosavljevic A, Ren B, Stamatoyannopoulos J A, Wang T, Kellis M. Integrative analysis of 111 reference human epigenomes. Nature. 2015; 518 (7539):317-30. Epub 2015 Feb. 20. doi: 10.1038/nature14248. PubMed PMID: 25693563; PMCID: PMC4530010.
3. Rao S, Han A L, Zukowski A, Kopin E, Sartorius C A, Kabos P, Ramachandran S. Mapping Transcription Factor-Nucleosome Dynamics from Plasma cfDNA. bioRxiv [Preprint]. 2021:2021.04.14.439883. doi: 10.1101/2021.04.14.439883.
4. Ramachandran S, Ahmad K, Henikoff S. Transcription and Remodeling Produce Asymmetrically Unwrapped Nucleosomal Intermediates. Mol Cell. 2017; 68 (6):1038-53 e4. doi: 10.1016/j.molcel.2017.11.015. PubMed PMID: 29225036.
5. Tumeh P C, Harview C L, Yearley J H, Shintaku I P, Taylor E J, Robert L, Chmielowski B, Spasic M, Henry G, Ciobanu V, West A N, Carmona M, Kivork C, Seja E, Cherry G, Gutierrez A J, Grogan T R, Mateus C, Tomasic G, Glaspy J A, Emerson R O, Robins H, Pierce R H, Elashoff D A, Robert C, Ribas A. PD-1 blockade induces responses by inhibiting adaptive immune resistance. Nature. 2014; 515 (7528):568-71. Epub 2014 Nov. 28. doi: 10.1038/nature13954. PubMed PMID: 25428505; PMCID: PMC4246418.
6. Haragan A, Gosney J R. Immunohistochemistry for prediction of response to immunotherapy. Diagnostic Histopathology. 2020.
7. Garon E B, Rizvi N A, Hui R, Leighl N, Balmanoukian A S, Eder J P, Patnaik A, Aggarwal C, Gubens M, Horn L, Carcereny E, Ahn M J, Felip E, Lee J S, Hellmann M D, Hamid O, Goldman J W, Soria J C, Dolled-Filhart M, Rutledge R Z, Zhang J, Lunceford J K, Rangwala R, Lubiniecki G M, Roach C, Emancipator K, Gandhi L, Investigators K-. Pembrolizumab for the treatment of non-small-cell lung cancer. N Engl J Med. 2015; 372 (21):2018-28. Epub 2015 Apr. 22. doi: 10.1056/NEJMoa1501824. PubMed PMID: 25891174.
8. Reck M, Rodriguez-Abreu D, Robinson A G, Hui R, Csoszi T, Fulop A, Gottfried M, Peled N, Tafreshi A, Cuffe S, O'Brien M, Rao S, Hotta K, Leiby M A, Lubiniecki G M, Shentu Y, Rangwala R, Brahmer J R, Investigators K-. Pembrolizumab versus Chemotherapy for PD-L1-Positive Non-Small-Cell Lung Cancer. N Engl J Med. 2016; 375 (19):1823-33. Epub 2016 Oct. 11. doi: 10.1056/NEJMoa1606774. PubMed PMID: 27718847.
9. Ventola C L. Cancer Immunotherapy, Part 3: Challenges and Future Trends. P T. 2017; 42 (8):514-21. Epub 2017 Aug. 7. PubMed PMID: 28781505; PMCID: PMC5521300.
10. Choi J, Baldwin T M, Wong M, Bolden J E, Fairfax K A, Lucas E C, Cole R, Biben C, Morgan C, Ramsay K A, Ng A P, Kauppi M, Corcoran L M, Shi W, Wilson N, Wilson M J, Alexander W S, Hilton D J, de Graaf C A. Haemopedia RNA-seq: a database of gene expression during haematopoiesis in mice and humans. Nucleic Acids Res. 2019; 47 (D1):D780-D5. Epub 2018 Nov. 6. doi: 10.1093/nar/gky1020. PubMed PMID: 30395284; PMCID: PMC6324085.
11. Cancer Genome Atlas Research N. Comprehensive molecular profiling of lung adenocarcinoma. Nature. 2014; 511 (7511):543-50. Epub 2014 Aug. 1. doi: 10.1038/nature13385. PubMed PMID: 25079552; PMCID: PMC4231481.
12. Clouthier D L, Lien S C, Yang S Y C, Nguyen L T, Manem V S K, Gray D, Ryczko M, Razak A R A, Lewin J, Lheureux S, Colombo I, Bedard P L, Cescon D, Spreafico A, Butler M O, Hansen A R, Jang R W, Ghai S, Weinreb I, Sotov V, Gadalla R, Noamani B, Guo M, Elston S, Giesler A, Hakgor S, Jiang H, McGaha T, Brooks D G, Haibe-Kains B, Pugh T J, Ohashi P S, Siu L L. An interim report on the investigator-initiated phase 2 study of pembrolizumab immunological response evaluation (INSPIRE). J Immunother Cancer. 2019; 7 (1):72. Epub 2019 Mar. 15. doi: 10.1186/s40425-019-0541-0. PubMed PMID: 30867072; PMCID: PMC6417194.
13. Chen J, Lopez-Moyado I F, Seo H, Lio C J, Hempleman L J, Sekiya T, Yoshimura A, Scott-Browne J P, Rao A. NR4A transcription factors limit CAR T cell function in solid tumours. Nature. 2019; 567 (7749):530-4. Epub 2019 Mar. 1. doi: 10.1038/s41586-019-0985-x. PubMed PMID: 30814732; PMCID: PMC6546093.

Whereas specific embodiments of the present inventive concept have been shown and described, it will be understood that other modifications, substitutions and alternatives are apparent to one of ordinary skill in the art. Such modifications, substitutions and alternatives can be made without departing from the spirit and scope of the inventive concept, which should be determined from the appended claims.

1. A method of identifying a disease state in a subject comprising:
sequencing cell-free DNA (cfDNA) derived from the subject;
obtaining a map of subnucleosomes at promoters associated with a map of TF binding sites through the sequencing of cfDNA; and
determining whether the subject has the disease or disorder if the map of subnucleosomes at promoters associated with the map of TF binding sites for the subject matches a signature for an individual having the disease or disorder.

2. The method of claim 1, wherein the subject is determined to be free of disease or disorder if the map of subnucleosomes at promoters associated with the map of TF binding sites for the subject matches a signature for an individual that is free of disease or disorder.

3. The method of claim 2, wherein the signature for an individual that is free of disease or disorder comprises a map of subnucleosomes at promoters associated with a map of TF binding sites in lymphoid and myeloid cells.

4. The method of claim 1, wherein the signature for an individual having the disease or disorder comprises a map of subnucleosomes at promoters associated with the map of TF binding sites in cells associated the disease or disorder.

5. The method of claim 1, wherein the disease or disorder is a cancer.

6. The method of claim 5, wherein the cancer is breast cancer.

7. (canceled)

8. The method of claim 1, wherein the map of TF binding sites comprises a map of FOXA1 binding sites.

9. The method of claim 1, wherein the map of TF binding sites comprises a map of estrogen receptor (ER) binding sites.

10. (canceled)

11. The method of claim 1, wherein the sequencing of cfDNA is performed on a single stranded cfDNA sequencing library derived from the subject.

12. The method of claim 11, wherein sequencing performed on the single stranded cfDNA sequencing library comprises:
identifying unique length profiles associated with different states and structures of nucleosomes and chromatosomes; and
obtaining a map of cfDNA fragments identifying transcription start sites,
wherein the transcription start sites to which the cfDNA fragments associated with subnucleosomes map provide a map of TF binding and a map of gene expression.

13. The method of claim 12, wherein cfDNA associated with TF binding has a fragment length distribution of less than about 147 basepairs.

14. The method of claim 1, wherein determining whether the subject has the disease or disorder comprises comparing the map of subnucleosomes at promoters and TF binding sites for the subject to a map of subnucleosomes at promoters and TF binding sites for a healthy individual and a map of subnucleosomes at promoters and TF binding sites for an individual having a disease or disorder.

15. The method of claim 1, wherein the signature for an individual having a disease or disorder comprises a map of subnucleosomes at promoters associated with a map of TF binding sites in cells from a patient-derived xenograft (PDX).

16. The method of claim 15, wherein the PDX is breast cancer PDX.

17-22. (canceled)

23. A method of monitoring efficacy or progress of treatment for a disease in a subject in need thereof comprising:
sequencing cell-free DNA (cfDNA) derived from a subject undergoing treatment for a disease or disorder;
obtaining a map of subnucleosomes at promoters associated with a map of TF binding sites through the sequencing of cfDNA; and
determining whether treatment of the subject is effective if the map of subnucleosomes at promoters associated with the map of TF binding sites for the subject starts to approximate a signature for an individual that is free of the disease or disorder.

24. The method of claim 23, wherein the signature for an individual that is free of disease or disorder comprises a map of subnucleosomes at promoters associated with a map of TF binding sites in lymphoid and myeloid cells.

25. The method of claim 23, wherein the subject is determined to require further, or alternate, treatment if the map of subnucleosomes at promoters associated with the map of TF binding sites matches a signature for the individual having, or still having, the disease or disorder.

26. The method of claim 23, wherein the disease or disorder is a cancer.

27. The method of claim 26, wherein the cancer is breast cancer.

28. (canceled)

29. The method of claim 23, wherein the map of subnucleosomes at promoters and TF binding sites comprises a map of FOXA1 binding sites.

30. The method of claim 23, wherein the map of subnucleosomes at promoters and TF binding sites comprises a map of estrogen receptor (ER) binding sites.

31. (canceled)

32. The method of claim 23, wherein the sequencing of cfDNA is performed on a single stranded cfDNA sequencing library derived from the subject.

33. The method of claim 32, wherein sequencing performed on the single stranded cfDNA sequencing library comprises:
identifying an enrichment of cfDNA fragments associated with subnucleosomes over fragments associated with nucleosomes and/or chromatosomes; and
mapping the cfDNA fragments identified to transcription start sites,
wherein the transcription start sites to which the cfDNA fragments associated with subnucleosomes map provide a map of TF binding and a map of gene expression.

34. The method of claim 33, wherein cfDNA associated with TF binding has a fragment length distribution of less than about 147 base pairs.

35. The method of claim 23, wherein determining whether treatment of the subject is effective comprises comparing the map of TF binding to a map of TF binding for an individual that is free of the disease or disorder and a map of TF binding for an individual having the disease or disorder.

36. The method of claim 23, wherein the signature for an individual having a disease or disorder comprises a map of subnucleosomes at promoters associated with a map of TF binding sites in cells from a patient-derived xenograft (PDX).

37. The method of claim 36, wherein the PDX is breast cancer PDX.

38-39. (canceled)

40. A method of monitoring recurrence of a disease or disorder in a subject in need thereof comprising:
sequencing cell-free DNA (cfDNA) derived from the subject;
obtaining a map of TF binding sites and subnucleosomes at promoters associated with the TF binding sites from the sequencing of cfDNA; and
determining whether the subject is having a recurrence of the disease or disorder if the map of subnucleosomes at promoters and TF binding sites for the subject matches a signature for an individual having the disease or disorder.

41-70. (canceled)
